(19) United States
(12) Patent Application Publication
(10) Pub. No.: US 2026/0242880 A1
(43) Pub. Date: Aug. 20, 2026

(54) DIAGNOSTIC ASSAYS AND METHODS FOR DETECTING PATHOGENS

(71) Applicants: Rutgers, The State University of New Jersey, New Brunswick, NJ (US); THE TRUSTEES OF PRINCETON UNIVERSITY, Princeton, NJ (US)

(72) Inventors: Padmapriya P. Banada, Edison, NJ (US); Nisha Modi, Hewitt, TX (US); Alexandra Bell, Princeton, NJ (US); Owen R. Dunkley, Palo Alto, CA (US); Yingda L. Xie, Jersey City, NJ (US); Cameron A. Myhrvold, Princeton, NJ (US)

(21) Appl. No.: 19/539,436

(22) Filed: Feb. 13, 2026

Related U.S. Application Data
(60) Provisional application No. 63/758,472, filed on Feb. 14, 2025.

(51) Int. Cl. C12Q 1/70; C12N 15/10; C12Q 1/34; C12Q 1/6806; C12Q 1/6823; C12Q 1/689

(52) U.S. Cl. CPC C12Q 1/70; C12N 15/1006; C12Q 1/34; C12Q 1/6806; C12Q 1/6823; C12Q 1/689; G01N 2333/922

(57) ABSTRACT

Provided are methods for co-extraction of DNA or RNA from two or more pathogens (such as Mycobacterium tuberculosis (Mtb) and SARS-CoV-2 (SC2)) in a sample. Also provided are assays for identifying a pathogen (such as Tuberculosis (TB)) directly from a sample.

### CROSS-REFERENCE TO RELATED APPLICATIONS

This application is entitled to priority pursuant to 35 U.S.C. § 119(e) to U.S. Provisional Application No. 63/758,472, filed on Feb. 14, 2025. The content of the application is incorporated herein by reference in its entirety.

REFERENCE TO AN ELECTRONIC SEQUENCE LISTING

The contents of the electronic sequence listing (096747.00500SeqList.xml; Size: 175,060 bytes; and Date of Creation: Feb. 12, 2026) is herein incorporated by reference in its entirety.

### STATEMENT REGARDING FEDERALLY SPONSORED RESEARCH OR DEVELOPMENT

This invention was made with government support under Grant Nos. AI168808, AI182281, TR003017, GM148739, and GM007388 awarded by the National Institutes of Health and grant number 753-0122-15113 awarded by the Centers for Disease Control and Prevention. The government has certain rights in the invention.

### FIELD OF THE INVENTION

This invention relates to methods for detecting two or more pathogens in a sample and assays for identifying a pathogen (such as Tuberculosis) in the sample.

### BACKGROUND

Tuberculosis (TB) and COVID-19 are two of the most lethal infectious diseases globally, each affecting millions every year. TB is caused by Mycobacterium tuberculosis (Mtb), an acid-fast bacteria, while COVID-19 is caused by coronavirus SARS-CoV-2 (SC2). COVID-19 has resulted in 7.1 million deaths worldwide and continues to be a major health challenge as new variants emerge and a total of 10.8 million people is estimated to have developed TB in 2023, with 8.2 million diagnosed and 1.25 million deaths reported. TB and COVID-19 continue to be a large public health crisis in the world especially in endemic countries, where medical resources, treatment, and community interventions are lacking. The global efforts toward eliminating TB were largely set back due to the COVID-19 pandemic leading to the detrimental rise of undiagnosed TB cases due to failed public health intervention programs. This, in turn, led to inefficient screening and management of the influx of co-infected MTB and SC2 cases, highlighting the need for easy and accessible multi-pathogen surveillance approaches implementable in community clinics for effective management and control of epidemics.

COVID-19 and TB are respiratory diseases affecting the lungs and can be diagnosed from respiratory samples. Current TB diagnostic methods are limited by trade-offs among sensitivity, turnaround time, and accessibility. Culture-based methods are very sensitive, but the turnaround time is slow (weeks). Conversely, sputum smear microscopy is rapid (~1 hour turnaround), but has limited sensitivity and specificity, in addition to relying on expert training. PCR-based tests, such as GeneXpert® MTB/RIF, are sensitive, but they require specialized equipment with an integrated thermocycler and proprietary single-use cartridges. Thus, there remains a critical unmet need for sensitive point-of-care TB diagnostics in low-resourced areas.

Sputum for pulmonary TB and nasopharyngeal/nasal/saliva for SC2 has been used frequently as preferred samples of choice in most diagnostics. Sputum is difficult to procure from Mtb patients especially in vulnerable populations (HIV positive, children, elderly) and often multiple sample collections or invasive procedures are needed to get a good quality clinical sputum sample, necessitating alternative sample types for diagnostics. Different sample types have been explored over the years including saliva, which has shown promise for pulmonary TB diagnosis with comparable sensitivity to sputum in lab-developed tests. Similarly, sputum samples showed highly comparable or better sensitivity to oropharyngeal samples for COVID-19 testing.

Nucleic acid-based (NA) molecular diagnostic tests have significantly improved TB and COVID-19 diagnosis in terms of time, sensitivity, specificity and overall cost effectiveness compared to traditional methods. There are a variety of NA-based tests, but there is a continued gap in reaching the diagnostic yield due, for example, to the sample type requirement for a particular test or inefficient extraction of nucleic acids limiting the test's performance. Therefore, there is a need for rapid, efficient, and less complicated tests that do not require costly equipment or kits.

### SUMMARY

In one aspect, provided is a dual assay for detecting a pathogen in a sample, comprising a first Cas-based assay and a second Cas-based assay comprising:
amplifying a target nucleic acid in the sample using recombinase polymerase amplification (RPA) to obtain an RPA product;
performing the first Cas-based assay by mixing a first Cas protein and a first CRISPR RNA (crRNA) with the RPA product in a first container, and performing the second Cas-based assay by mixing a second Cas protein and a second crRNA with the RPA product in a second container.

In some embodiments, the target nucleic acid comprises at least two different nucleic acid sequences. In some embodiments, the target nucleic acid comprises DNA or RNA. In some embodiments, the target nucleic acid comprises Mycobacterium tuberculosis (Mtb) insertion element IS6110 or IS1081.

In one embodiment, the first Cas-based assay and the second Cas-based assay are performed in parallel. In some embodiments, the first Cas protein and the second Cas protein is Cas12a or Cas13a. In one embodiment, Cas12a is from Lachnospiraceae bacterium. In one embodiment, Cas13a is from Leptotrichia wadei.

In one embodiment, the pathogen comprises Mtb. In one embodiment, the sample is from a subject infected by Mtb.

In some embodiments, the crRNAs comprise any one of the sequences shown in Table 8 or Table 9. In some embodiments, the RPA is performed using one or more primers set forth in Table 8 or Table 9.

In some embodiments, the assay comprises analyzing products of the first Cas-based assay and the second Cas-based assay using a lateral flow assay or a fluorescence-based assay. In some embodiments, the first Cas protein, the second Cas protein, the first crRNA, or the second crRNA is lyophilized.

In some embodiments, the sample comprises sputum or saliva. In one embodiment, the sample is derived from a tongue swab. In one embodiment, the subject is a human.

In some embodiments, detection of the pathogen is completed within 90 minutes or less.

In some embodiments, the target nucleic acid is obtained by:
a) adding a chelating agent to chelate polyvalent metal ions in the sample and a lysis agent to lyse two or more pathogens in the sample;
b) vortexing the sample;
c) heating the sample at about 95° C. for at least 30 minutes; and
d) spinning the sample to collect supernatant containing the nucleic acid,
   wherein the target nucleic acid is obtained in 15 minutes or less.

In one aspect, provided is a kit comprising:
a container comprising a set of recombinase polymerase amplification (RPA) primers, a recombinase, and a crowding agent, wherein the container is adapted to receive a target nucleic acid;
two different containers, each of which comprises a Cas protein and a CRISPR RNA (crRNA), wherein the two containers are adapted to perform a Cas-based assay; and optionally, instructions for use.

In one aspect, provided is a method of co-extracting nucleic acid from two or more pathogens in a sample from a subject infected or suspected of being infected by the two or more pathogens, comprising:
a) adding a chelating agent to chelate polyvalent metal ions in the sample and a lysis agent to lyse the two or more pathogens in the sample;
b) vortexing the sample;
c) heating the sample at about 95° C. for at least 30 minutes; and
d) spinning the sample to collect supernatant containing the nucleic acid, wherein the method is completed in 15 minutes or less.

In some embodiments, the two or more pathogens comprise Mycobacterium tuberculosis (Mtb) and SARS-CoV-2 (SC2). In one embodiment, the lysis agent comprises lysing matrix B beads. In one embodiment, the chelating agent comprises Chelex-100 resin. In some embodiments, the nucleic acid comprises RNA and DNA.

In some embodiments, the sample is vortexed for at least 30 seconds.

In some embodiments, the sample is heated at about 95° C. for at least 30 minutes such as at least 30 minutes, 31 minutes, 32 minutes, 33 minutes, 35 minutes, 40 minutes, etc.

In some embodiments, the sample comprises saliva or sputum. In one embodiment, the sample is derived from a tongue swab. In one embodiment, the sample is from a human.

In one embodiment, the supernatant is analyzed with a diagnostic test. In some embodiments, the diagnostic test is real-time PCR (qPCR), digital PCR (dPCR), or a CRISPR-based diagnostic.

### DESCRIPTION OF DRAWINGS

FIGS. 1A and 1B illustrate optimization of chelex-resin based sample processing. FIG. 1A is a graph of the genomic copy numbers of the indicated samples. Chelex-100 was supplemented with various detergents, and total DNA was extracted by boil prep and the eluant was tested with digital PCR. FIG. 1B is a graph of the relative fluorescent units of the indicated samples. Chelex-100 was supplemented with various detergents, and total DNA was extracted by boil prep and the eluant was tested with a SHINE-TB Cas13a assay. Sp. Sputum; PC-Positive control ATCC Mtb H37Ra at 10³ copies/reaction; NTC: No template control.

FIGS. 2A-2E demonstrate saliva sample processing and eluant compatibility across various platforms. FIG. 2A is a schematic of the dynamic range for total DNA extracted using the sample processing protocol with saliva tested in real-time PCR (acr gene) in Roche LC480. FIG. 2B is a graph of the results using QIAcuity® dPCR (IS6110). FIG. 2C is a graph of the genomic copy numbers using a SHINE-TB Cas13a assay (IS6110) in Cytation 3 platform showing real-time curves. FIG. 2D is a graph of the end point fluorescence units at 90 min using a SHINE-TB Cas13a assay (IS6110) in a Cytation 3 platform. FIG. 2E is a graph of the relative fluorescence units of the indicated samples. Sp. Sputum; PC-Positive control ATCC Mtb H37Ra (used at 10 copies/reaction in dPCR); EC—non-spiked saliva extraction control; NTC: No template reaction control.

FIGS. 3A-3F illustrate sputum sample processing and reduction of sample inhibition in SHINE-TB assay. FIG. 3A is a graph of the relative fluorescent units of the indicated sputum samples. Chelex Resin based Boil prep (CRB) sample processing to extract total DNA was tested with various negative patient sputum samples spiked with M. bovis BCG (10⁵ CFU/mL; a vaccine strain) and was tested with a SHINE-TB assay. FIG. 3B is a graph of the genomic copy numbers of the indicated samples. Optimization of the CRB method in dPCR was used supplementing with lysing matrix beads (LMB). FIG. 3C is a graph showing the evaluation of BCG spiked (10⁵ CFU/mL) individual patient sputum samples with the optimized CRB+LMB method. FIG. 3D is a graph of re-evaluation of a SHINE-TB assay with BCG spiked individual (10⁵ CFU/mL) patient sputum samples with an optimized CRB+LMB method. FIG. 3E is a graph of the effect of dilution on sputum samples using Tris-EDTA buffer evaluated with a SHINE-TB assay. FIG. 3F is a graph of the evaluation of inhibitory samples using the TE dilution method. FIGS. 3A, 3D, 3E, and 3F: SHINE-TB test end point fluorescence units (λ490-517) at 90 min.

FIGS. 4A-4E show sputum sample processing protocol evaluation with a single copy target M. bovis BCG IS6110. FIG. 4A is a schematic of the final optimized sputum sample processing protocol. FIG. 4B is a graph of the dynamic range for total DNA extracted from spiked pooled sputum tested in real-time PCR targeting IS6110 gene in Roche LC480. FIG. 4C is a graph of the genomic copy numbers of the sputum spiked with M. bovis BCG using QIAcuity® dPCR targeting IS6110. In particular, shown are real time curves (X490-517). FIG. 4D is a graph of the end point fluorescence units (X490-517) at 90 min using a SHINE-TB Cas13a assay in a tested Cytation3 platform. PC-Positive control M. bovis BCG (at 2000 copies/reaction); EC—Extraction negative control; NTC: No template reaction control.

FIGS. 5A-5D illustrate co-detection of Mtb (10⁵ CFU/ml) and SARS-CoV-2 (10⁵ PFU/ml) in salivary sputum. FIG. 5A is a graph of the relative fluorescent units of the indicated samples using CRISPR Cas13 assays for TB. FIG. 5B is a graph of the relative fluorescent units of the indicated samples using CRISPR Cas13 assays for SC2. FIG. 5C is a graph of the genomic copy numbers (Mtb gene IS6110) for the indicated samples using dPCR for TB. FIG. 5D is a graph of the genomic copy numbers (SC2 N1 gene) using dPCR for SC2. PC-Positive control M. bovis BCG (at 2000 copies/reaction) or SARS-CoV-2 (SC2) WT strain WA1; EC—Extraction negative control; NTC: No template reaction control.

FIGS. 6A-6F depict a CRISPR-Cas12a assay for detecting Mtb. FIG. 6A is a schematic showing the flow of the reaction. DNA target is amplified using primers, recombinases, strand-displacing polymerase, and ssDNA binding proteins. This may be directly detected by Cas12a or turned into RNA with T7 reverse transcription for detection with Cas13a. FIG. 6B is a graph of the fluorescent read-out at 90 minutes of Cas12a detecting synthetic DNA for six different crRNA primer designs. Amplification was allowed to proceed for 30 minutes before the addition of crRNA and Cas12a. Units are in copies/μL. FIG. 6C is a graph of the fluorescent read-out at 90 minutes of Cas12a detecting synthetic targets with varying concentrations of Cas12a. Molar concentration alone indicates that all components were present from the start of the reaction. Molar concentration spiked indicates that amplification was allowed to proceed for 30 minutes before the addition of crRNA and Cas12a. Units are in copies/μL. FIG. 6D is a graph of the fluorescent read-out at 90 minutes of Cas12a detecting synthetic targets. All components were present from the start of the reaction. Units are in copies/μL. FIG. 6E is a graph of the performance of the Cas12a assay for decreasing concentrations of H37Rv (mc²6230; an auxotroph) genome. Units are in genomes/μL. FIG. 6F is a graph of the cross-reactivity panel for Cas12a assay against various nontuberculous mycobacteria (NTMs) at 1 ng/μL. In FIGS. 6B and 6D, error bars: SD based on n=3 replicates. In FIGS. 6C, 6E, and 6F, error bars: SD based on n=2 replicates.

FIGS. 7A-7D illustrate CRISPR-Cas13a based detection of Mtb. FIG. 7A is a graph of the fluorescent read-out at 90 minutes of Cas13a detecting synthetic DNA for six different crRNA-primer designs against two different targets, IS6110 and IS1081. Units are in copies/μL. FIG. 7B is a graph of the fluorescent read-out at 90 minutes of comparison of Cas12a to Cas13a detection of IS6110 synthetic DNA. Cas13a is a single-guide assay for more direct comparison. Units are in copies/μL. FIG. 7C is a graph of the fluorescent read-out at 90 minutes of Cas13a detecting H37Rv in single-target detection and dual-target detection formats. Units are in genomes/μL. FIG. 7D is a graph of the cross-reactivity panel for the Cas13a dual detection assay against various NTMs and other pathogens at 1 ng/μL. Error bars: SD based on n=3 replicates.

FIGS. 8A-8G show validation of the Cas13a dual detection assay against clinically relevant samples. FIG. 8A is a graph of the fluorescent readout of Cas13a detecting BCG spiked into sputum: TE matrix and Cas12a detecting internal control. The Cas13a reporter is FAM whereas the Cas12a reporter is HEX. Units are in CFU/mL. FIG. 8B is a graph of the fluorescent readout of fresh Cas13a (left) or lyophilized Cas13a (right) detecting synthetic internal control and BCG genome. Synthetic targets were mixed at a concentration of 104 and 103 copies/μL, respectively. FIG. 8C is a lateral Flow read-out of Cas13a detecting IS6110+IS1081 (top) and Cas12a detecting internal control (bottom) in BCG spiked into sputum:TE matrix. Appearance of the top line is a positive readout. Faint lines are interpreted as negative. Disappearance of the bottom line is not necessary. FIG. 8D is a graph of the results of a range finding experiment demonstrating the dynamic range of the fresh Cas13a dual detection assay. Cas13a detecting BCG and Cas12a detecting internal control. BCG culture (left) and BCG spiked into sputum:TE (right). FIG. 8E is a graph of the results of testing fresh Cas13a and Cas12a coupled assay against TB-negative clinical samples. Numbers indicate patient number. Fluorescent read-out at 90 minutes. FIG. 8F is a graph of the fresh limit of detection (LoD) for H37Rv. LOD=69.0 (51.0-86.9). FIG. 8G Fresh LoD for BCG. LOD=80.5 (59.4-101.6). In FIGS. 8A, 8B, 8D, and 8E, error bars: SD based on n=3 replicates. FIG. 8C shows only one replicate. FIG. 8F has 20 replicates of all concentrations and FIG. 8G has 21 replicates of all concentrations.

FIGS. 9A-9D show TB detection from sputum using fresh SHINE-TB. FIG. 9A is a schematic showing the workflow of patient sample testing. Sputum samples were collected and tested using GeneXpert® in Colombia. Samples were shipped to Rutgers for novel sample processing. Processed samples were used for CRISPR assays and dPCR. FIG. 9B is a graph of TB detection in 14 different sputum samples using Cas13a dual detection. For each sample, a line is shown at the mean of 3 technical replicates. Shading: Shading for sputum, matrix only, and NTC samples—Controls, Shading from samples 001 to 008 and 008 to 014—correct call, Shading for sample 007—unmatched call for GeneXpert® vs CRISPR and culture. LoD is shown as a dotted line at 2900 a.u. Triplicates were averaged for determination of positive and negative. For culture, plus sign means positive detection, minus sign means no detection. GeneXpert® ND means no detection. FIG. 9C depicts tables showing Cas13a dual detection compared to culture (left), GeneXpert® (middle left), and dPCR (middle right). Table showing GeneXpert® vs Culture (right). Further details are in Table 6. FIG. 9D is a graph of average Cas13a (FAM) vs Cas12a internal control (HEX) signal for each sample seen in FIG. 9B. Linear trend was determined for the positive samples with an R squared value of 0.7058.

FIG. 10 depicts a Cas13 crRNA and primer set. IS6110 Cas13 guide set (top) and IS1081 Cas13 guide set (bottom). Portions of consensus sequence top and bottom strand are shown, 5′ prime and 3′ prime ends are labeled in gray. Reverse primer is labeled in gray, all nucleotides that bind are shown with dashes. Forward primer is labeled in gray, all nucleotides that bind are shown with dashes. Portions of forward primer that do not bind are part of the T7 promoter. T7 promoter allows for IS6110 amplicon to be transcribed into RNA (labeled in red). crRNA is labeled in gray, all nucleotides that bind are shown with dashes. Portions of the crRNA that do not bind are the direct repeat.

FIG. 11 shows a IS6110 Cas12 guide set. Portions of consensus sequence top and bottom strand are shown, 5′ prime and 3′ prime ends are labeled in gray. Reverse primer is labeled in gray, all nucleotides that bind are shown with dashes. Forward primer is labeled in red, all nucleotides that bind are shown with dashes. crRNA is labeled in gray, all nucleotides that bind are shown with dashes. Portions of the crRNA that do not bind are the direct repeat.

FIG. 12 shows LTR5 internal control guide set. Portions of consensus sequence top and bottom strand are shown, 5′ prime and 3′ prime ends are labeled in gray. Reverse primer is labeled in gray, all nucleotides that bind are shown with dashes. Forward primer is labeled in gray, all nucleotides that bind are shown with dashes. crRNA is labeled in gray, all nucleotides that bind are shown with dashes. Portions of the crRNA that do not bind are the direct repeat.

### DETAILED DESCRIPTION

This disclosure provides assays that address a critical unmet need for sensitive point-of-care TB diagnostics in low-resourced areas. Isothermal amplification methods have the potential to bridge the accessibility and accuracy gap by eliminating the need for thermocycling in a sequence-specific signal amplification protocol. Loop-mediated isothermal amplification (LAMP) is most widely used within this category but requires incubation at 65° C. for the duration of the reaction and is known to occasionally produce false positive results because it relies on numerous long primers that each have a non-negligible probability of off-target hybridization. Recombinase polymerase amplification (RPA) can amplify sequences at 37° C. using only a pair of primers per amplicon, but can still be subject to non-specific amplification when longer primers are used for more efficient reactions. Thus, a second layer of sequence specificity gating is needed to reduce false positive signals.

This disclosure also relates to methods for co-extracting nucleic acid from two or more pathogens in a sample and assays, for example, for detecting Tuberculosis (TB). In one embodiment, the two or more pathogens comprise Mycobacterium tuberculosis (Mtb) and SARS-CoV-2 (SC2).

The methods for co-extracting nucleic acid from two or more pathogens in a sample as described herein address a long-felt need of developing a rapid and simple approach for diagnosing infectious diseases. One advantage of the co-extraction methods described herein is that they bypass the need for specialized equipment, which streamlines multi-pathogen testing. Notably, the co-extracted nucleic acid is suitable for application in various diagnostic assays.

CRISPR-based diagnostics (CRISPR-Dx) leverage the high specificity and collateral cleavage activities of CRISPR-associated proteins such as Cas12 and Cas13 that help avoid high levels of background signal and false positives inherent to the nucleic acid amplification techniques with which they are often paired. Cas12 is an RNA-guided DNA endonuclease that recognizes DNA complementary to its guide CRISPR RNA (crRNA) (Chen et al. 2018) and requires a protospacer adjacent motif for double-stranded DNA recognition (Zetsche et al. 2015). Upon recognition, Cas12 cleaves its target in cis and cleaves nearby single-stranded DNA in trans (Chen et al. 2018; Li et al. 2018). In contrast, Cas13 targets and cleaves RNA in both cis and trans (Abudayyeh et al. 2016). Both Cas12 and Cas13 require substantial crRNA-target complementarity to shift the nucleases to their active forms, making reporter cleavage events highly specific to the presence of a target sequence (Tambe et al. 2018; Abudayyeh et al. 2016; Chen et al. 2018). Two representative orthologs, Cas12a and Cas13a, both exhibit high turnover catalytic activity, and pairing them with an excess of cleavable nucleic acid reporter provides substantial signal amplification, fundamentally enhancing sensitivity compared to directly quantifying the products of isothermal amplification. The enhancements owed to recognition-gated high catalytic turnover thus enable reliable detection with enhanced specificity and sensitivity compared to isothermal amplification techniques alone.

Several groups have employed CRISPR-Dx paired with PCR or isothermal amplification to address accessibility issues in TB diagnosis. However, these methods require user manipulations and specialized instrumentation between the preamplification of the sample and detection, making these assays hard to deploy and exposing the workflow to potential sources of contamination. Peng and colleagues developed a Cas12-based format that combines amplification and detection, but requires instrument-based nucleic acid extraction (Peng et al. 2024; Peng et al. 2024). Therefore, the assays described herein address an unmet need for CRISPR-Dx methods designed to work in constrained environments with simplified sample preparation techniques for the detection of tuberculosis-causing mycobacteria without specialized instrumentation.

### I. Dual Assay for Detecting a Pathogen

In one aspect, provided is a dual assay for detecting a pathogen in a sample, comprising a first Cas-based assay and a second Cas-based assay comprising:
amplifying a target nucleic acid in the sample using recombinase polymerase amplification (RPA) to obtain an RPA product;
performing the first Cas-based assay by mixing a first Cas protein and a first CRISPR RNA (crRNA) with the RPA product in a first container; and
performing the second Cas-based assay by mixing a second Cas protein and a second crRNA with the RPA product in a second container.

RPA begins when T4 UvsX binds to primers in the presence of ATP and a crowding agent (a high molecular weight polyethyleneglycol), forming a recombinase-primer complex. The crowding agent prevents spontaneous recombinase-primer disassembly that occurs in the presence of the single-stranded binding proteins needed for the amplification. The complex then interrogates double-stranded DNA seeking a homologous sequence and promotes strand invasion by the primer at the cognate site. The displaced DNA strand is stabilized by single-stranded binding proteins. Then the recombinase disassembles and a strand displacing DNA polymerase (e.g., large fragment of Bacillus subtilis Pol 1, Bsu) binds to the 3′ end of the primer to elongate it in the presence of dNTPs. This process undergoes cyclic repetition, resulting in the amplification of a given target (e.g., DNA or RNA). RPA operates at temperatures ranging from 22° C. to 45° C. such as 22° C., 25° C., 27° C., 29° C., 30° C., 32° C., 34° C., 36° C., 38° C., 40° C., 42° C., 44° C., 45° C., or any temperature therebetween. In particular, RPA can amplify as few as 1-10 DNA target copies in under 20 minutes (See Lobato and O'Sullivan. Trends Analyt. Chem. 2018. 98: 19-35).

As used herein, the term “RPA product” refers to the amplified DNA from the RPA reaction.

As used herein, the term “pathogen” refers to an organism, including a microorganism, which causes disease in another organism (e.g., animals and plants) by directly infecting the other organism, or by producing agents that cause disease in another organism (e.g., bacteria that produce pathogenic toxins and the like). As used herein, pathogens include, but are not limited to bacteria, protozoa, fungi, nematodes, viroids and viruses, or any combination thereof, wherein each pathogen is capable, either by itself or in concert with another pathogen, of eliciting disease in vertebrates including but not limited to mammals, and including but not limited to humans. As used herein, the term “pathogen” also encompasses microorganisms which may not ordinarily be pathogenic in a non-immunocompromised host. Nonlimiting examples of viral pathogens include Herpes simplex virus (HSV)1, HSV2, Epstein Barr virus (EBV), cytomegalovirus (CMV), human Herpes virus (HHV) 6, HHV7, HHV8, Varicella zoster virus (VZV), hepatitis C, hepatitis B, adenovirus, Eastern Equine Encephalitis Virus (EEEV), West Nile virus (WNV), JC virus (JCV), and BK virus (BKV). Other examples of pathogens include, but are not limited to Mycobacterium tuberculosis (Mtb) and SARS-CoV-2 (SC2).

In some embodiments, target nucleic acid comprises at least two different nucleic acid sequences. In some embodiments, the target nucleic acid comprises DNA or RNA. In some embodiments, the target DNA comprises Mycobacterium tuberculosis (Mtb) insertion element IS6110 or IS1081.

In some embodiments, one Cas-based assay serves as a positive internal control to mitigate false negatives caused by inhibitors, salivary dilution, or poor nucleic acid extraction. In one embodiment, the Cas-based assay that serves as a positive control targets the Long Terminal Repeat (LTR) of the endogenous retrovirus ERVK.

In some embodiments, the first Cas-based assay and the second Cas-based assay are performed sequentially. In a preferred embodiment, the first Cas-based assay and the second Cas-based assay are performed in parallel.

As used herein, the term “Cas protein” refers to an enzyme produced by the CRISPR system. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof.

In some embodiments, the first Cas protein and the second Cas protein is Cas12a or Cas13a. In one embodiment, Cas12a is from Lachnospiraceae bacterium. In one embodiment, Cas13a is from Leptotrichia wadei. In one embodiment, the first Cas protein and the second Cas protein are the same. In one embodiment, the first Cas protein and the second Cas protein are different.

In one embodiment, the Cas12β-based assay serves as a positive internal control. In one embodiment, the Cas12a-based internal control targets the LTR of the endogenous retrovirus ERVK.

In some embodiments, the pathogen includes, but is not limited to Mtb. In one embodiment, the sample is from a subject infected by Mtb. In one embodiment, the subject is a human.

In some embodiments, the crRNAs comprise any one of the sequences shown in Table 8 or Table 9. In some embodiments, the RPA is performed using one or more primers set forth in Table 8 or Table 9.

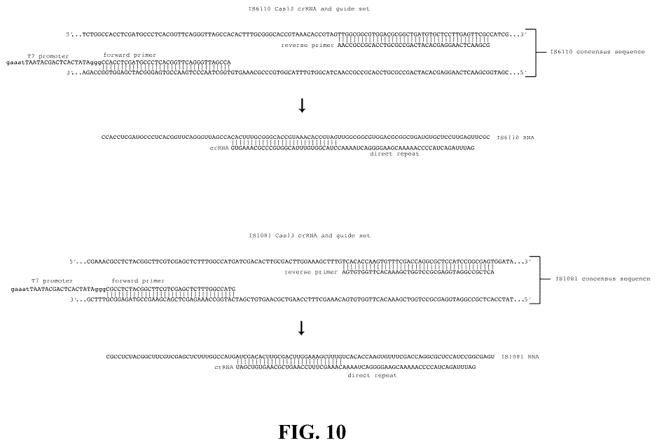

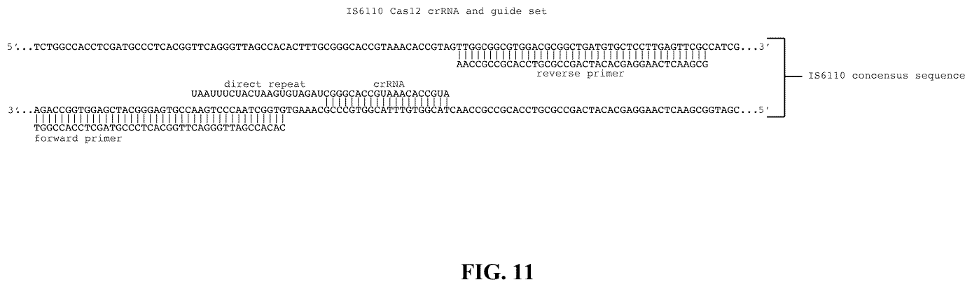

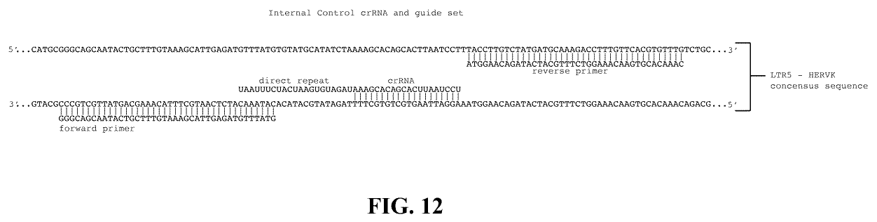

A representative Cas13crRNA and primer set is shown in FIG. 10. A representative IS6110 Cas12 guide set is shown in FIG. 11. A representative LTR5 internal control guide is shown in FIG. 12.

In some embodiments, the assay comprises analyzing products of the first Cas-based assay and the second Cas-based assay using a lateral flow assay or a fluorescence-based assay as known in the art. As used herein, the term “lateral flow assay” refers to a paper-based test that detects and measures a target substance (such as nucleic acid) in a sample.

In one embodiment, the first Cas protein, the second Cas protein, the first crRNA, or the second crRNA is lyophilized. In some embodiments, the first Cas protein, the second Cas protein, the first crRNA, or the second crRNA is lyophilized into single-use tubes.

In some embodiments, the sample comprises sputum or saliva. As used herein, the term “sputum” refers to a mixture of saliva and mucus.

In some embodiments, the sample comprises wastewater. Also provided are methods of using the assay described herein for environmental surveillance (such as wastewater surveillance).

For example, detecting a pathogen in wastewater can indicate the prevalence of an infectious disease within a given geographical area.

In some embodiments, the detection of the pathogen is completed within 90 minutes or less such as 90 minutes, 85 minutes, 80 minutes, 75 minutes, 70 minutes, 65 minutes, 60 minutes, etc. In one embodiment, the detection of the pathogen is completed within 30 minutes or less such as 30 minutes, 25 minutes, 20 minutes, 15 minutes, 10 minutes, 5 minutes, etc. In some embodiments, the detection of the pathogen is completed within 5 minutes to 90 minutes such as 5 minutes, 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, or any time therebetween.

In one embodiment, the target nucleic acid is obtained by:
a) adding a chelating agent to chelate polyvalent metal ions in the sample and a lysis agent to lyse two or more pathogens in the sample;
b) vortexing the sample;
c) heating the sample at about 95° C. for at least 30 minutes; and
d) spinning the sample to collect supernatant containing the nucleic acid,
   wherein the target nucleic acid is obtained in 15 minutes or less such as 15 minutes, 14 minutes, 13 minutes, 12 minutes, 11 minutes, 10 minutes, etc.

### Kit

In one embodiment, provided is a kit comprising:
a container comprising a set of recombinase polymerase amplification (RPA) primers, a recombinase, and a crowding agent, wherein the container is adapted to receive a target nucleic acid;
two different containers, each of which comprises a Cas protein and a CRISPR RNA (crRNA), wherein the two containers are adapted to perform a Cas-based assay; and
optionally, instructions for use.

In some embodiments, the Cas protein includes, but is not limited to Cas12a or Cas13a. In some embodiments, the crRNA comprises a sequence as shown in Table 8 or Table 9. In some embodiments, the RPA primers comprise a sequence as shown in Table 8 or Table 9. In some embodiments, the target nucleic acid comprises DNA or RNA. In some embodiments, the target nucleic acid comprises Mtb insertion element IS6110 or IS1081.

II. Co-Extracting Nucleic Acids from Two or More Pathogens

In one aspect, provided is a method of co-extracting nucleic acid from two or more pathogens in a sample from a subject infected or suspected of being infected by the two or more pathogens, comprising:
a) adding a chelating agent to chelate polyvalent metal ions in the sample and a lysis agent to lyse the two or more pathogens in the sample;
b) vortexing the sample;
c) heating the sample at about 95° C. for at least 30 minutes; and
d) spinning the sample to collect supernatant containing the nucleic acid,
   wherein the method is completed in 15 minutes or less.

In some embodiments, the two or more pathogens comprise Mycobacterium tuberculosis (Mtb) and SARS-CoV-2 (SC2). In one embodiment, the lysis agent comprises lysing matrix B beads. In one embodiment, the chelating agent comprises Chelex-100 resin.

As used herein, the term “chelating agent” refers to chemicals that form molecules with certain metal ions, inactivating the ions so that they cannot react with other elements, thus a binding agent that suppresses chemical activity by forming chelates. Chelation is the formation or presence of two or more separate bindings between a ligand and a single central atom. The ligand may be any organic compound, a silicate or a phosphate.

Examples of chelating agents include, but are not limited to EDTA (ethylene diamine tetraacetate), NTA (2,2′,2″-nitrilotriacetate), citrate, 2-hydroxypropan-1,2,3-tricarboxylate, DTPA (diethylenetriaminepentaacetic acid), MGDA (methylglycinediacetic acid or N,N”-bis(carboxymethyl)alanine), EGTA (ethylene glycol tetraacetic acid), EDDS (ethylenediamineN,N′-disuccinic acid), GLDA (L-Glutamic acid, N,N-diacetic acid), Polycarboxylates such as PAA [poly(acrylic acid)], PAA/PMA [copoly(acrylic acid/maleic acid)], sodium tripolyphosphate (STP), HEDP (1-Hydroxyethylidene-1,1-Diphosphonic Acid), EDTMP [bis(phosphonomethyl)amino]methylphosphonic acid] or (ethylenediamine tetra(methylene phosphonic acid)), EDTMPA (ethylenediaminetetramethylenetetraphosphonic acid), DTPMP (diethylenetriamine penta (methylene phosphonic acid), and DTMPA (diethylenetriaminepenta(methylenehosphonic acid)). Chelating agents may contain nitrogen such as in EDTA, NTA, DTPA, PDTA, GLDA, MGDA, EDDS, EDTMP, EDTMPA, and DTPMP or ASMA, ASDA, ASMP, IDA, SMAS, SEAS, SMGL, SEGL, MIDA, α-ALDA, SEDA, ISDA, PHDA, ANDA, SLDA, TUDA, SMDA, HEDTA, DEG, ATMP, or mixtures thereof.

In one embodiment, the nucleic acid comprises RNA and DNA. In some embodiments, the nucleic acid comprises Mtb insertion element IS6110 or IS1081.

In one embodiment, the sample is vortexed for at least 30 seconds, such as at least 30 seconds, 31 seconds, 32 seconds, 33 seconds, 34 seconds, 35 seconds, 36 seconds, 37 seconds, 38 seconds, 39 seconds, 40 seconds, 45 seconds, 50 seconds, 55 seconds, 60 seconds, etc.

In one embodiment, the supernatant is analyzed with a diagnostic test. The diagnostic test includes, but is not limited to real-time PCR (qPCR), digital PCR (dPCR), or a CRISPR-based diagnostic (such as the CRISPR-based diagnostic described in Section II). In one embodiment, the CRISPR-based diagnostic test is SHINE-TB (Streamlined Highlighting of Infections to Navigate Epidemics). SHINE-TB addresses the demand for rapid, sensitive, and accessible diagnostics for TB (Arizti-Sanz et al. 2022; Arizti-Sanz et al. 2020). SHINE-TB consists of two parallelized single-pot reactions that combine isothermal amplification by RPA, in vitro transcription, and detection, including a Cas13a assay for detecting two conserved elements in the Mtb genome and a Cas12a assay for detecting human DNA as an internal control.

In some embodiments, the sample is saliva or sputum. In one embodiment, the sample comprises saliva or sputum. In one embodiment, the sample is derived from a tongue swab. In one embodiment, the sample is from a human.

As used herein, the term “crowding agent” refers to an agent that prevents the spontaneous recombinase-primer disassembly that occurs in the presence of the single-stranded binding proteins needed for the amplification. Examples of a crowding agent include, but are not limited to polyethylene glycol (PEG), dextran, and ficoll.

As used herein, the term “in vitro” refers to events that occur in an artificial environment, e.g., in a test tube or reaction vessel, in cell culture, etc., rather than within a multi-cellular organism.

As used herein, the term “subject” refers to a mammal. The term “mammal” as used herein is intended to include, but is not limited to, humans, laboratory animals, domestic pets, and farm animals. Mammals include, but are not limited to, a human or non-human mammal, such as a canine, bovine, equine, ovine, or feline, etc. Individuals and patients are also subjects herein.

As used herein, the term “container” refers to an object that is used to hold something (such as reagents for a reaction). Examples of containers include, but are not limited to test tubes, multi-well plates, or Eppendorf tubes.

As used herein, the singular forms “a,” “an,” and “the” include plural references unless the context clearly dictates otherwise.

As used herein, the terms “including,” “comprising,” “containing,” or “having” and variations thereof are meant to encompass the items listed thereafter and equivalents thereof as well as additional subject matter unless otherwise noted.

As used herein, the phrases “in one embodiment,” “in various embodiments,” “in some embodiments,” and the like are used repeatedly. Such phrases do not necessarily refer to the same embodiment, but they may unless the context dictates otherwise.

As used herein, the terms “and/or” or “/” mean any one of the items, any combination of the items, or all of the items with which this term is associated.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1. Materials and Methods for Simplified Co-Extraction of Total Nucleic Acids from Respiratory Samples for Detection of Mycobacterium tuberculosis and SARS-CoV-2 Optimized for Compatibility Across Diagnostic Platforms

This Example describes the materials and methods used in Examples 2-4.

### 1A. Bacterial Cultures

An attenuated strain of M. tuberculosis H37Rv (mc²6230, Mtb), and a vaccine strain M bovis BCG (BCG) were used in all the experiments herein unless indicated. Both were cultured in BD DIFCO™ 7H9 Middlebrook broth supplemented with 10% Middlebrook OADC growth supplement (BD) and 0.05% Tween 80 (Sigma Aldrich, St Louis, MO) as per the manufacturer's recommendations (BD, Franklin Lakes, NJ). M. tuberculosis H37Rv (mc²6230), has independent deletions in the panC and panD genes and requires media supplemented with 24 μg/ml of Calcium Pantheonate (Sigma Aldrich, St Louis, MO). Both strains were then grown to an optical density OD of 0.6-0.8 and sub-cultured twice more until the OD reached 0.6-0.8 again. The grown culture stock was mixed, aliquoted and stored at −80° C. until further use. For spiking experiments, an aliquot of the stock culture was thawed at 4° C., sonicated for 30 s (Branson CPX1800-E Ultrasonic water bath, Danbury, CT) and rested for 30 s on ice. This was repeated twice and placed on ice for an additional six minutes. Ten-fold serial dilutions were performed in 7H9 and were quantified on Middlebrook 7H10 agar plates (BD).

### 1B. Clinical Samples

Both saliva and sputum were used as a matrix for this evaluation. Samples were obtained from 39 participants at the University Hospital (UH) and Lattimore Clinic at the International Center for Public Health in Newark, NJ, between May 2022 and November 2023. All participants were US-based individuals unsuspected for TB because their clinical presentation was not consistent with Tuberculosis disease. They were diagnosed with other respiratory conditions (COPD, etc.) and non-respiratory conditions (liver disease, fractures, etc.). All clinical data, including gender, age, and underlying conditions, were obtained, and recorded. Samples were characterized based on physical properties, logged, aliquoted, and stored at −80° C. until use. The use of the terminology “salivary sputum” refers to 1:1 diluted saliva in sputum. All individual sputum samples were tested by IS6110-dPCR for TB negativity before spiking with Mtb CFUs.

### 1C. Optimization of Sample Processing

Sample processing buffer was designed using Chelex-100 resin as the base reagent. Chelex-100 was supplemented with various detergents such as Nonidet P-40 (NP-40, Sigma), Tween20 (Sigma), Tergitol (Sigma), and TritonX (Sigma) at 1% concentration, for enhanced nucleic acid recovery. Negative saliva obtained from BioIVT from 4 known, Mtb-negative participants (confirmed by Xpert® MTB/Rif Ultra Test) was pooled at equal volumes and mixed via snap vortexing. Sputum collected from discarded patient samples from a Microbiology lab at the University Hospital, Newark, NJ were pooled separately based on physical characteristics to evaluate the variability of the sputum sample type. The pools were verified negative by Xpert® Ultra Test. Mtb was spiked at ~10⁶ CFU/ml saliva/sputum matrix.

The initial protocol involved adding 200 μl of the chelex based buffer to 100p of spiked saliva/sputum in a screw-cap Eppendorf tube with an O-ring. The samples were vortexed for 30 s at 3400 rpm, incubated for 30 min at 95° C., and then centrifuged for 2 min at 9400×g. The supernatant containing the nucleic acid was carefully collected (~100 μl). All the extracted samples were analyzed via the qPCR IS6110 assay, dPCR IS6110 assay and the CRISPR-Cas13a IS6110/IS1081 assay.

### 1E. Assays Used for Validation

The QIAcuity® ProbePCR kit (Qiagen, Hilden, Germany) was used for the QIAcuity® Digital PCR System. The reaction components varied based on the nanoplate type, but the standardized kit-recommended reaction mix and cycling conditions were utilized. For 8.5K nanoplates (Qiagen, Hilden, Germany), the 12 μl reaction mix was comprised of 3 μl of PCR Master Mix (Qiagen, Hilden, Germany), 0.8 uM of each primer, 0.4 μM of probe, 5.8 μl of water; and 2 μl of DNA. For 26K nanoplates (Qiagen, Hilden, Germany), the 40 μl reaction mix was comprised of 10 μl of ProbePCR Master Mix, 0.8 μM of each primer, 0.4 μM of probe, 5.8 μl of water; and 2 μl of DNA. The dPCR cycling conditions included an initial heat activation at 95° C. for 2 minutes, then 40 cycles of denaturation at 95° C. for 15 seconds, and annealing/extension at 60° C. for 30 seconds.

Real-time PCR was performed in the LightCycler 480 system, in a 384-well microliter plate at a reaction volume of 12 μl (using the QIAcuity® probe PCR kit), comprised of 3 μl of ProbePCR Master Mix (Qiagen, Hilden, Germany), 0.8 uM of each primer, 0.4 μM of IS6110 assay probe, 5.8 μl of water, and 2 μl of DNA. The PCR cycling conditions included an initial heat activation at 95° C. for 2 minutes, then 40 cycles of denaturation at 95° C. for 15 seconds, and annealing/extension at 60° C. for 30 seconds and extension at 72° C., 15 seconds. The fluorescence was recorded during the annealing step of the assay.

Primer and probe sequences are shown in Table 1. CRISPR SHINE-TB test was performed using the single-step Cas13a assay described herein and CRISPR SHINEv.2 SARS-CoV-2 test (Arizti-Sanz J, et al. 2022. Simplified Cas13-based assays for the fast identification of SARS-CoV-2 and its variants. Nature Biomedical Engineering 6:932-943). Briefly, for SHINE-TB test master mix containing SHINE buffer (20 mM HEPES pH 8.0, 60 mM KCl, 3.5% PEG-8000, nuclease-free (nf) water), 45 nM LwaCas13a (Genscript, stored in 100 mM Tris HCl pH 7.5 and 1 mM DTT), 0.3 nM of each rNTP (New England Biolabs), 1 U/mL T7 RNAP (Biosearch Technologies), 62.5 nM FAM 6U quenched reporter (5′-/56-FAM/rUrUrUrUrUrU/3IABkFQ/-3′; IDT), 22.5 nM of each crRNA in a combination, 70 nM of the appropriate RPA primers in nf water and finally, 14 nM MgOAc. Assays were performed by mixing completed Cas13a master mix (90% by volume) with target (10% by volume). Reactions were loaded in technical triplicates into 384-well clear-bottom microplates (Greiner) for 15 μL reactions or 20 μL reactions in 384-well microplates (Corning). The plates were incubated at 37° C. for up to 3 hours in an Agilent BioTek Cytation 5 microplate reader, with fluorescence readings programmed to record every 5 minutes.

TABLE 1
Primers and probes.

| | | | | 5′ | | 3′ |
|---|---|---|---|---|---|---|
| | | | | Modifi- | | Modifi- |
| | Reference | | | cation | Sequence | cation |
| dPCR | IS6110 | | Primer | | CGCCGCTTCGGACCACCAGCA | |
| | gene | | Forward | | C (SEQ ID NO: 1) | |
| | | | Primer | | GTGACAAAGGCCACGTAGGCG | |
| | | | Reverse | | AACC (SEQ ID NO: 2) | |
| | | Chakravorty, | Probe | FAM | CGGCTGTGGGTAGCAGACCTC | BHQ1 |
| | | 201 | | | ACC (SEQ ID NO: 3) | |
| qPCR | acr | | Primer | | CTAATACCGGATAGGACCACG | |
| | gene | | Forward | | G (SEQ ID NO: 4) | |
| | | | Primer | | CTCATCCCACACCGCTAAAGC | |
| | | | Reverse | | G (SEQ ID NO: 5) | |
| | | Malherbe, | Probe | FAM | CGCTCCCCTTCGTTCGCACGG | DABCYL |
| | | 2016 | | | TGTCGCTGGAGCG (SEQ ID | |
| | | | | | NO: 6) | |
| dPCR | N1 | | Primer | | GACCCCAAAATCAGCGAAAT | |
| | gene | | Forward | | (SEQ ID NO: 7) | |
| | | | Primer | | TCTGGTTACTGCCAGTTGAAT | |
| | | | Reverse | | CTG (SEQ ID NO: 8) | |
| | | Lu, | Probe | FAM | ACCCCGCATTACGTTTGGTGG | BHQ1 |
| | | 2020 | | | ACC (SEQ ID NO: 9) | |

### 1F. Extraction Efficiency

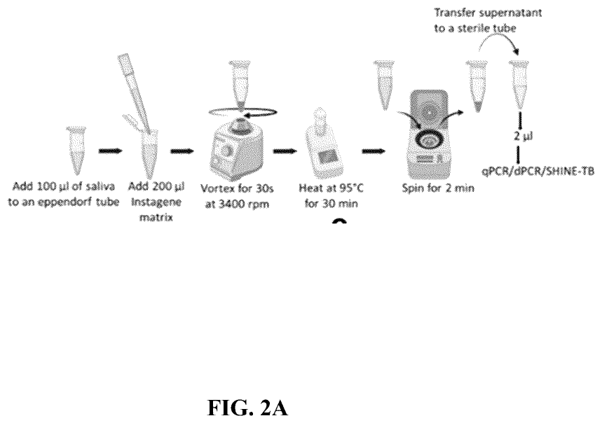

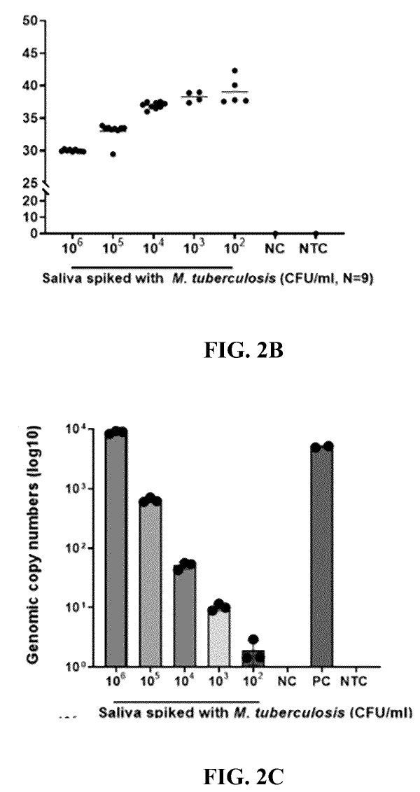

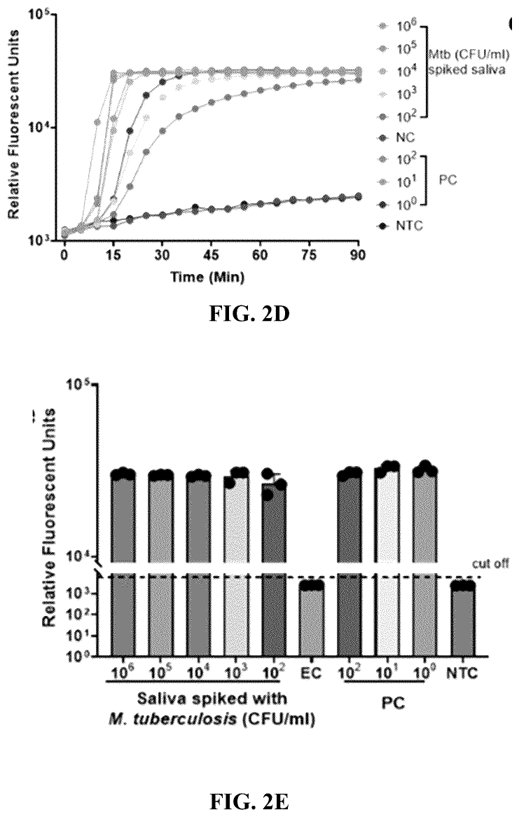

M. bovis BCG was spiked to pooled sputum at different concentrations at 10⁵, 10⁴, 10³ and 10 CFU/ml and total nucleic acids was extracted following the protocol described in FIG. 2A. The dPCR was performed using IS6110 assay following the method described herein.

### Example 2. Optimization of Chelex-100 Resin Based Sample Processing for Saliva and Sputum

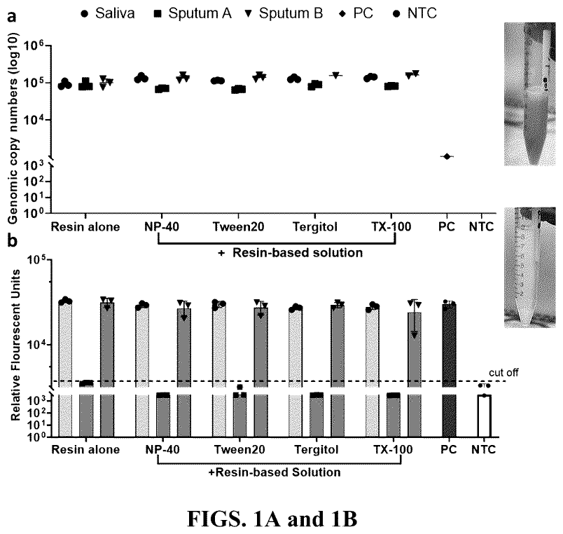

To evaluate the effect of different detergents that are often used to improve extraction yield from bacteria, the Chelex-100 resin was supplemented with various detergents, and tested individually from saliva and sputum samples spiked with Mtb at 10⁶ CFU/ml. Evaluation of the DNA extracted using the protocol as described in the methods, using dPCR showed no significant difference between Chelex-100 with and without detergents for both saliva and two different kinds of sputum pools A and B (FIG. 1A). With the CRISPR SHINE-TB assay Sputum B worked equivalent to saliva in all conditions (FIG. 1B). However, sputum A, which was more colored and mucoidal than sputum B, was inhibitory to the test across all solutions (FIG. 1B). Considering the natural biological variation to be expected with sputum samples, further testing and optimization was necessary to efficiently detect Mtb from sputum in CRISPR-TB based tests.

### Example 3. Mtb Spiked Saliva Sample Processing and Detection

Confirmed negative saliva sample pool (BioIVT) was spiked with Mtb H37Rv Mc²6230 at logarithmic dilutions ranging from 10⁶ CFU to 100 CFU/ml. Mtb DNA was extracted from saliva using the optimized chelex resin-based boil prep (CRB) protocol as described in FIG. 2A. The extracted DNA from the dynamic range concentrations were tested in variety of Mtb assays and in different platforms showed compatibility across real-time PCR targeting the Mtb acr gene in Roche LC480 (FIG. 2B), dPCR in QIAcuity® targeting Mtb IS6110 gene (FIG. 2C), and in CRISPR SHINE-TB test (FIGS. 2D and 2E). The detection sensitivity varied between different assays, where the acr assay (a single copy gene) showed a sensitivity of 100% (9/9) at >104 CFU/ml, whereas below 50% at 1000 (4/9) and 100 (5/9) CFU/ml of Mtb spiked in saliva. However, dPCR and SHINE-TB tests targeting IS6110 showed sensitivity of 100% at 100 CFU/ml (FIGS. 2A-2E). Thus, the Mtb DNA sample from saliva eluted using the protocol described herein produced an inhibitor-free DNA, which is applicable in a broad range of platforms and with different Mtb assays.

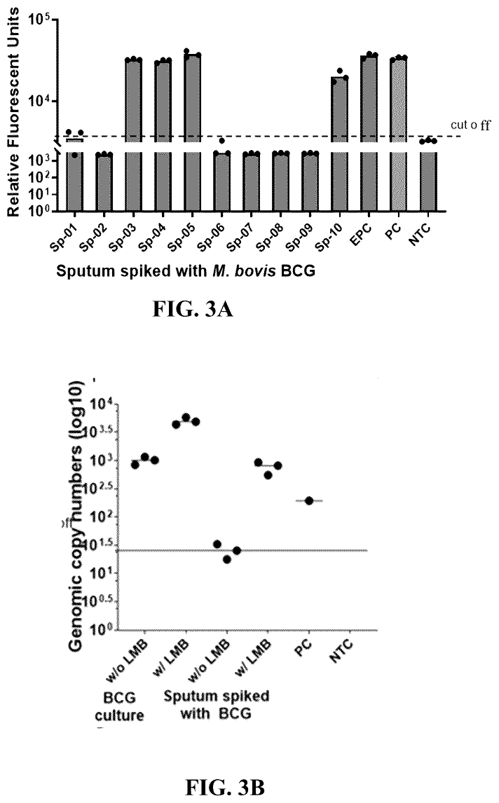

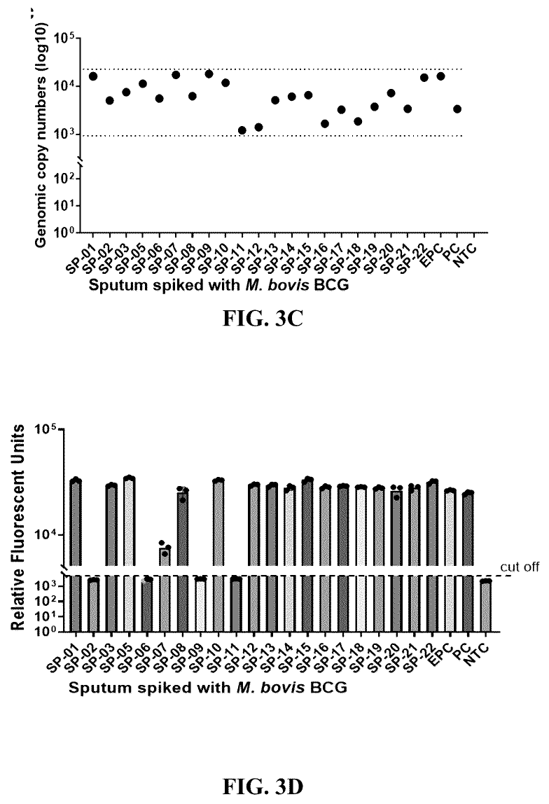

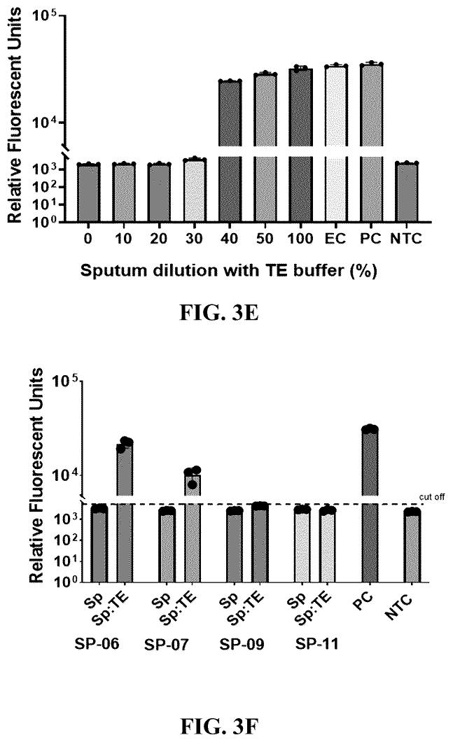

Sputum is widely used and accepted sample type for TB testing, which was further evaluated using the CRB protocol (FIG. 2A). From FIGS. 1A and 1B, it was conclusive that the test performance might be affected by variations in the composition of sputum samples. Thus, 10 individual sputum samples were collected from 10 different TB unsuspected patients using SHINE-TB test (FIG. 3A), where 5 out of 10 samples could not be detected at 10⁵ CFU/ml of M bovis BCG. It was suspected that either 1) insufficient mycobacterial lysis or 2) presence of CRISPR specific inhibitors (cas or RPA) was the cause. To address insufficient lysis, ready-to-use 2 ml tubes were used containing lysing matrix B beads (LMB, MP Biomedicals), which are 0.1 mm silica spheres to enhance the Mtb lysing efficacy. M. bovis BCG was spiked at 10⁵ CFU/ml to the sputum pooled with hardy sputa (samples that did not work: Sp-02, Sp-06, Sp-07, Sp-08 and Sp-09) and DNA was extracted following the modified CRB protocol (FIG. 2A) where LMB tubes were used instead of Eppendorf tubes and a 2 μl of the extract was tested in dPCR. As shown in FIG. 3B, a 2-log 10 increase in copy number of BCG was observed in spiked sputum using LMBs compared to without, indicating the positive influence of the beads on improved lysing resulting in higher concentrations of DNA. Further testing in dPCR with 21 individual sputum samples spiked with BCG showed consistently higher detection using LMBs where all samples (21/21) were detected by dPCR, however the genomic copies ranged from 1208 to 18100, indicating extraction variability from sample to sample. When the same 21 sample set was tested in CRISPR SHINE-TB test, 17/21 (81%) were detected and the remaining 4 samples Sp-02, Sp-06, Sp-09, and Sp-11 were not detected. Extraction efficiency does not seem to be the problem for this sample set failure since these samples had >10³ genomic copies as established by dPCR and the established LoD of SHINE-TB test is <100 CFU/ml. Except for Sp-11 which showed a lower copy number in dPCR (FIG. 3C) all other samples had ≥10⁴ genomic copies per reaction. Thus, it was suspected that the presence of RPA/cas enzyme specific inhibitors was the reason for assay failure. Using a simple approach of dilution with TE buffer, it was found that sample to TE buffer>40% (40 of TE buffer and 60 of sputum) started showing positivity with the SHINE-TB test (FIG. 3E), confirming this. A 1:1 dilution was selected for further testing with inhibitory sputa from FIG. 3D. TE dilution did show a positive influence with 50% of the samples testing positive (FIG. 3F). Commercially available Instagene matrix (Biorad) to lab prepared chelex powder at different concentrations was also compared with and without LMB and it was found that there was a significant difference between IGM vs 10% chelex using LMBs.

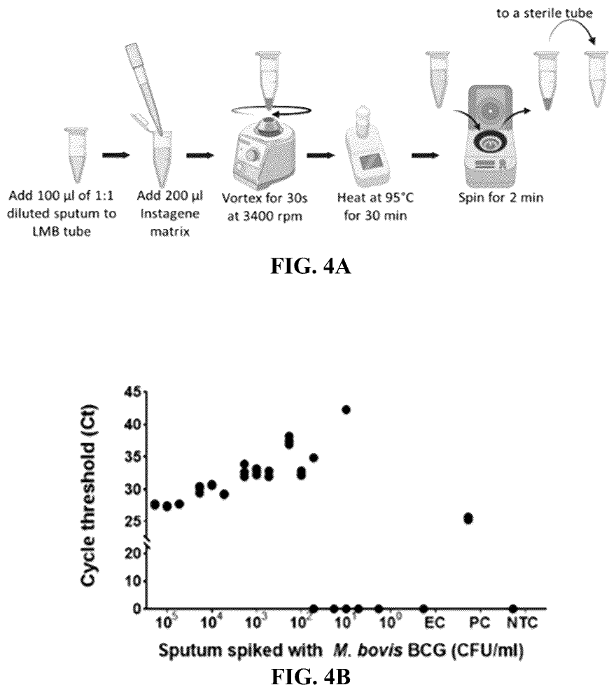

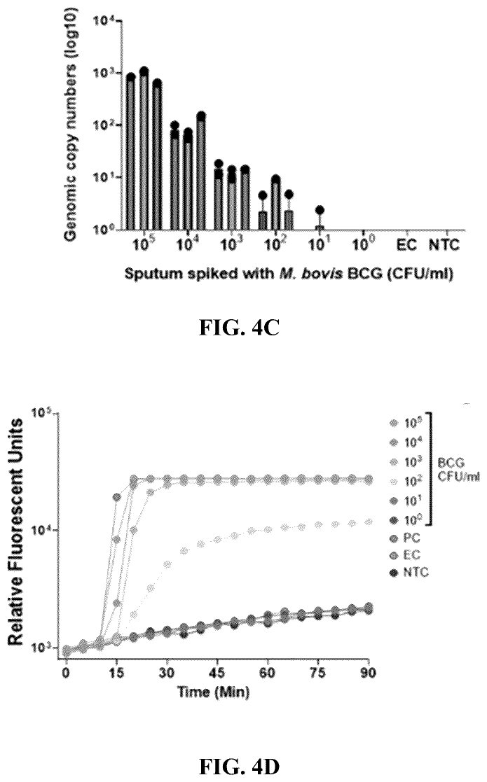

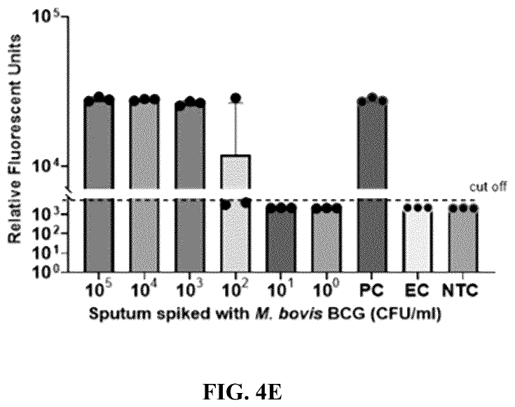

Using this simple boil prep in combination with lysis beads and dilution (CRB, FIG. 4A) it was established that the eluate from saliva/salivary sputum is compatible with a variety of platforms including qPCR (LC480), dPCR (QIAcuity®) and CRISPR-based (SHINE-TB) (FIGS. 4A-4E). Extraction efficiency calculated from this data where BCG was spiked to sputum at different concentrations, was found to correspond to 48% (10⁵ CFU/ml), 62.2% (10⁴ CFU/ml), 86.4% (10³ CFU/ml), and 99.3% (10 CFU/ml), with an average being ~74%.

### Example 4. Co-Extraction of Mtb and SARS-CoV-2 Spiked in a Single Salivary Sputum

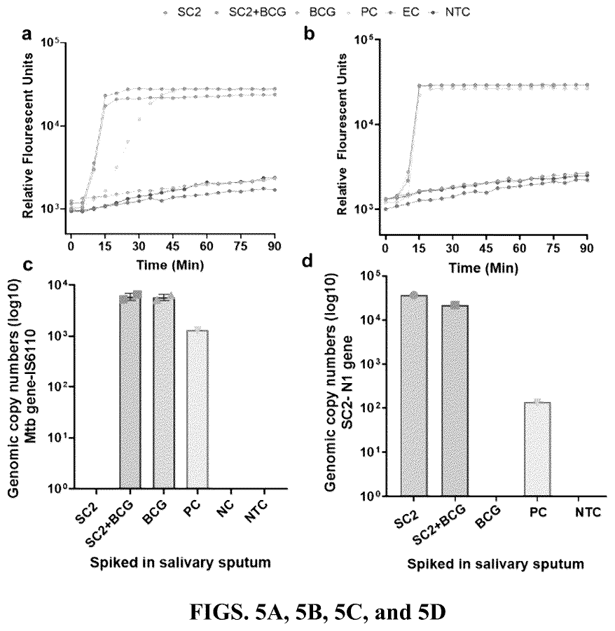

M. bovis BCG and inactivated SARS-CoV-2 USA WA1/2020 (SC2) strains were spiked at 10⁵ CFU/ml or 10⁵ PFU/ml to pooled negative salivary sputum samples at different conditions. A set of samples (N=3) contained only BCG, and a 2^{nd} set for only SC2, and a 3^{rd} set containing both organisms spiked at a 1:1 ratio. The samples were processed using the optimized protocol for sputum as described in FIG. 4A. All the samples were evaluated in both dPCR and CRISPR SHINE-TB test. As shown in FIGS. 5A-5D, both BCG DNA (FIGS. 5A and 5C) and SC2 RNA (FIGS. 5B and 5D), were efficiently extracted using the CRB protocol and detected using both dPCR and the SHINE-TB test.

The above-described results were optimized and resulted in the development of a simple, rapid and efficient sample processing method for total nucleic acid extraction from saliva and salivary sputum as a potential sample type for dual respiratory pathogen detection. This method utilizes Chelex-100 resin and a Lysing matrix tube with 0.1 mm-silica beads, combined with heating at 95° C., which demonstrated an extraction efficiency ranging from 48% (for 10⁵ CFU/ml) to as high as 99.3% (10 CFU/ml). Considering MTB is a hardy and difficult to lyse bacteria, most of the initial studies were focused on optimizing MTB lysis in both saliva and sputum. Sputum, which has been the primary specimen of choice for TB diagnosis has poised itself with a variety of challenges for NAAT based diagnostics. Other sample types such as saliva, oral swabs, and tongue swabs are promising alternatives for respiratory samples. Saliva, the primary specimen of choice for SC2 diagnosis, varies from patient to patient and can have many different physical characteristics, along with contaminants (blood, drug, food, tissue, etc.), which need careful optimization to extract pathogen nucleic acids at high efficiency, rapidity yet simple enough for applicability in resource limited settings. Nucleic acid extraction protocols for respiratory samples depend on the downstream applications. For most PCR based diagnostic assays crude extracts often suffice the need, whereas whole genome sequencing or transcriptomics etc., might need higher quality samples. For point-of-care diagnostic testing, simple and low-cost methods were screened that can be used by non-laboratory personnel. The procedures and their limitations were identified that overlap between standard viral and bacterial extraction methods and require minimal manipulation (Tables 1 and 2); ideally, steps that are utilized in the field currently. Chelex-100 resin and heating are commonly utilized for both SC2 and MTB.

The NA extract using the above-described sample processing method for saliva spiked with MTB was compatible across different platforms including LC480 qPCR, QIAcuity® digital PCR, and CRISPR SHINE-TB test detecting as low as 100 CFU/ml (FIGS. 2A-2E). However, the extract from MTB spiked in various MTB spiked sputum samples showed low compatibility at 82% positivity (18/22, FIG. 3D) compared to 100% with dPCR (FIG. 3C). Efforts to spike DNA to extracted negative matrix did not show any inhibitory effect and as high as 10% blood spiked in saliva did not cause any inhibition in the SHINE-TB test indicating efficient removal of most common inhibitors by the established CRB protocol. Thus, with TE buffer dilution at 1:1, 50% (2/4 tested) of the samples could be rescued. Other methods were also evaluated such as using mass-based filter tips to potentially isolate genomic material from large macromolecules (lipids, proteins, food, etc.) and various concentrations of Chelex-100 resin mixture (from 6% to 20%) with limited success. CRISPR-based diagnostic testing is emerging as a highly promising test with point of care (POC) potential for pathogen surveillance and strain identification and thus needs comprehensive evaluation and optimization for sputum sample processing for TB detection.

The capability of the protocol was also established in co-extracting total nucleic acids (DNA and RNA) from Mtb and SC2 using respective CRISPR SHINE tests for TB (Dunkley et al., 2024) and cov-2 (Arizti-Sanz J et al. 2020. Streamlined inactivation, amplification, and Cas13-based detection of SARS-CoV-2. Nature Communications 11:5921) (FIGS. 5A-5D) and using dPCR targeting N1 gene for SARS-CoV-2 and IS6110 for TB (FIGS. 5A-5D). This study is the first to establish a co-extraction protocol from a single sample.

TABLE 2
Review of existing methodologies for TB and SC2 nucleic acid extraction.

| | | | | |
|---|---|---|---|---|
| | | | | Limitations or |
| | Extraction | | Processing | different from the |
| No | method | Notes | time | method of interest |

For DNA extraction from Mycobacterium tuberculosis (MTB)

| | | | | | |
|---|---|---|---|---|---|
| 1 | Boil method | In this simple method, distilled | ~8 | min | Used for cultures not |
| | | water was added to the sputum | | | directly from |
| | | sample, mixed via vortex and placed | | | samples |
| | | in a boiling water bath (100° C.) for | | | High speed |
| | | 3 min. The mixture was then | | | centrifugation is |
| | | centrifuged at 15000 rpm for 5 min | | | needed. |
| | | at 4º C., and the supernatant was | | | |
| | | separated for DNA analysis using | | | |
| | | 1% agarose gel electrophoresis, | | | |
| | | PCR targeting ripA gene of MTB, | | | |
| | | and sequencing. | | | |
| 2 | TE Boil | This method involves addition of TE | ~20 | min | Used for sample |
| | extraction | buffer and placing the sample pellet | | | pellets, not directly |
| | | and TE mixture in boiling water bath | | | from the samples |
| | | for 15 min followed by centrifugation | | | requiring additional |
| | | at 16000 xg for 5 min. | | | processing. |
| | | The eluant was compatible | | | Need for specialized |
| | | with PCR. | | | instruments such as |
| 3 | IDI | TE buffer and pellet were placed | ~25 | min | a vortex genie |
| | extraction | into an IDI lysis tube which contains | | | Qiagen kits are |
| | | glass bead matrix. The tube was | | | expensive, and the |
| | | vigorously mixed for 5 min using | | | procedures are long. |
| | | Vortex Genie 2 and then placed in a | | | Requires some |
| | | boiling water bath. | | | technical skill. |
| 4 | Qiagen | In this method, authors used the | >1 | h | |
| | extraction | QIAGEN QIAmp DNA mini kit | | | |
| | | tissue protocol, but added enzymatic | | | |
| | | digestion steps with lysozyme. | | | |
| | | Boiled for 15 min, and proteinase K | | | |
| | | step was at 56° C. for 1 hour. Mean Ct | | | |
| | | values with the real time assays were | | | |
| | | like the TE boil extraction method, | | | |
| | | however, the yield was lower. | | | |
| 7 | GITC-boil | In this method, diverse samples | 3 | h | Very lengthy |
| | prep method | were preprocessed first in several | | | protocol, |
| | | different ways, involving several | | | Guanidium |
| | | reagents such as N-acetyl L-cysteine, | | | isothiocyanate |
| | | guanidinium isothiocyanate, | | | (GITC) is |
| | | Tris-HCl, EDTA, Sarcosyl, | | | hazardous, produces |
| | | and M β-mercaptoethanol, | | | toxic gas, and |
| | | and centrifugation, and 1-mm glass | | | difficult to discard |
| | | beads. After the inhibitor removal | | | easily. |
| | | step, Chelex-100 resin, Tween20 | | | Not compatible with |
| | | and Triton X-100 were added to the | | | point of care use |
| | | preprocessed samples. The mixture | | | with limited |
| | | was then heated at 90° C. for 40 min, | | | technical skills. |
| | | followed by centrifugation. This | | | GITC samples may |
| | | method was tested with various | | | not be compatible |
| | | pulmonary and extrapulmonary | | | with many non-PCR |
| | | samples. DNA samples were | | | based downstream |
| | | compatible with PCR. | | | applications |
| 8 | Chelex-100 | Sample culture pellet was incubated | ~30 | min | Culture pellet only. |
| | method | with 20% Chelex-100 resin. After | | | No direct sample. |
| | combined | vortex mixing, the mixture was | | | Extraction efficiency |
| | with boiling | placed for boiling at 100° C. for 15 | | | was similar to the |
| | and | min, and placed in ultrasonic water | | | method described |
| | sonication | bath for 15 min. DNA was separated | | | herein. |
| | | after 5 min centrifugation at 14000 g | | | Compatibility shown |
| | | and was analyzed based on IS6110 | | | only for PCR based |
| | | gene by real-time qPCR in the | | | assays. |
| | | LightCycler 480 system for | | | GITC is hazardous |
| | | efficiency. The proportion of MTB | | | and not compatible |
| | | recovered was 82% with this | | | with non-PCR based |
| | | method relative to the estimated | | | methods such as |
| | | input, and authors chose to evaluate | | | CRISPR based. |
| | | the clinical sputum samples with | | | |
| | | this method. | | | |
| 9 | Boiling | Incubate sample pellet with lysis | >1 | h | |
| | method | buffer containing Tris-HCl, EDTA, | | | |
| | combined | NaCl, and guanidium isothiocyanate | | | |
| | with | (GTIC) for 20 min, and apply it | | | |
| | Guanidinium | through 3 cycles of freeze thawing | | | |
| | Isothiocyanate | (−80º C. for 5 min and | | | |
| | | 100° C. for 5 min) and | | | |
| | | boiling at 100° C. for 15 min. | | | |
| | | DNA was analyzed for IS6110 gene | | | |
| | | in PCR. The proportion of MTB | | | |
| | | recovered was ~45% with this | | | |
| | | method relative to the estimated | | | |
| | | input. | | | |
| 10 | MagPurix | Samples were processed to get | 1 | h | Need for specialized |
| | TB DNA | pellets using NaLC-NaOH method. | | | kits and instruments. |
| | extraction | Compared Chelex-100 instagene | | | Requires some |
| | kit | matrix with MagPurix, a magnetic | | | technical skills. |
| | | particle separation technology and | | | |
| | | Magpurix 12s automated nucleic | | | |
| | | acid purification system using the | | | |
| | | manufacturer's recommendations. | | | |
| 11 | Mechanical | Different types of beads such as | ~22 | min | Pellets of samples |

| | | |
|---|---|---|
| disruption | 0.1 mm zirconia, 0.2 mm and 1 mm | need additional pre- |
| with beads | glass beads were mixed with sputum | processing. |
| followed by | sample pellet suspended in TE. The | |
| boiling and | mixture was vortexed for 5 min and | |
| Chelex | incubated at 95° C. for 5 min. | |
| extraction | 100 μL debris free lysate after | |
| | centrifugation was mixed with an | |
| | equal volume of 10% Chelex 100 | |
| | resins, incubated at room | |
| | temperature for 10 min, and | |
| | centrifuged at 1000 g for 2 min. | |
| | DNA was analyzed by multiplex | |
| | PCR targeting IS6110 and Rv0927c- | |
| | pstS3 regions of MTB in an Applied | |
| | Biosystems 7700 real-time system. | |
| | It was shown that smaller size glass | |
| | beads result in better extraction with | |
| | this method. | |

SARS-COV-2 RNA extraction

| 1 | Extraction | Saliva and oral swab samples were | ~55 | min | Lengthy, RT-PCR |
|---|---|---|---|---|---|
| | by heat | added with an equal volume of TE | | | testing only. |
| | fixation and/ | buffer or with Proteinase K, were | | | No other |
| | or using | incubated at room temperature for | | | downstream |
| | ProteinaseK | 10 min, followed by incubation at | | | applications |
| | | 98º C. for 30-45 min. Extracted | | | were tested. |
| | | RNAs were evaluated by RT-PCR | | | |
| | | test targeting E and RdRp genes. For | | | |
| | | saliva, this method shows 71.43% | | | |
| | | diagnostic sensitivity, vs 56.12% for | | | |
| | | dry swab. | | | |
| 2 | Acid | This extraction method uses a lysis | ~40 | min | NP specimens only |
| | pH-based | buffer with pH 5 and containing | | | Not so simple. |
| | method | SDS, sodium citrate dehydrate, | | | SDS containing |
| | | anhydrous citric acid, and EDTA. | | | samples are not |
| | | SDS lyses viral protein coats, and | | | always compatible |
| | | low pH helps in the recovery of | | | with downstream |
| | | RNA. After the lysis buffer was | | | applications. |
| | | added to the sample and mixed, | | | Requires many |
| | | precipitation buffer that also | | | chemicals and |
| | | contains sodium citrate dehydrate, | | | reagents and |
| | | anhydrous citric acid, and NaCl was | | | multiple |
| | | added. The following steps are | | | centrifugation steps, |
| | | incubation on ice, multiple | | | making this |
| | | centrifugations, and supernatant | | | impractical for use |
| | | recovery in isopropanol and 70% | | | in resource limited |
| | | ethanol, and pellet resuspension in | | | settings. |
| | | pre-warmed nuclease-free water. | | | |
| | | Nasopharyngeal samples underwent | | | |
| | | this extraction, and the extracted | | | |
| | | RNA was evaluated by one step RT- | | | |
| | | qPCR targeting RNAseP, N1 and N2 | | | |
| | | genes. This method gave high yield | | | |
| | | and comparable results with | | | |
| | | commercial extraction kit. | | | |
| 3 | Guanidinium | This method involves mixing the | ~19 | min | GITC based |

| | | |
|---|---|---|
| Isothiocyanate, | sample with three reagents: | methods are not |
| phenol, and | guanidinium isothiocyanate, phenol, | always compatible |
| chloroform- | and chloroform, vortexing, | with non-PCR based |
| based | incubation at room temperature, and | diagnostics. |
| extraction | centrifugation for 15 min at 12000 g. | Introduces |
| | RNAs were extracted from | additional chemical |
| | nasopharyngeal (NP) and | hazard and is |
| | oropharyngeal (OP) swab samples | difficult to easily |
| | and evaluated by one-step RT-qPCR | discard samples due |
| | targeting the N gene of SARS- | to potential toxic |
| | CoV2. The detection level with this | gases produced with |
| | method was ~4 copies/μL of RNA. | common |
| | | disinfectants like |
| | | bleach. |

### Example 5. Materials and Methods for a Streamlined Point-of-Care CRISPR Test for Tuberculosis Detection Directly from Sputum

This Example describes the materials and methods used in Examples 6-10.

### 5A. Synthetic Targets and Genomic DNA

Gene fragments for IS6110 and IS1081 were ordered from Integrated DNA Technologies (IDT) (sequences listed in Table 8) and resuspended in nuclease-free water. Bacille Calmette-Guerin (BCG) genomic DNA was ordered from ATCC (catalog 35734D-2) and quantified with dPCR. Quantitative Mtb genomic DNA was ordered from ATCC (25177DQ).

### 5B. Sequence Design

Cas12a target sequences were chosen by first compiling canonical sequences and related diversity information for chosen insertion sequences or possible internal control genes using BLAST (Altschul et al. 1990) and Clustal Omega (Madeira et al. 2024) on publicly available datasets. These sequences were annotated for all canonical and permitted non-canonical Cas12a PAM sequences, and ranked candidates manually for conservation throughout the amplified region and the energetics of crRNA ensemble conformations (Zadeh et al. 2011). Cas13a designs were generated using ADAPT (Metsky et al. 2022) on the same consensus sequences with 40 nt primers and otherwise default parameters, before being manually ranked for conservation and free energy, as above. The Cas13a guide IS6110 C had a slightly lower ADAPT rating than the other chosen candidates but was made to overlap with Cas12a cr5 to compare the two assays' efficiencies more directly. A T7 RNA polymerase promoter was added to the 5′ end of all forward primers for Cas13a assays (Table 8).

### 5C. Two-Step and One-Step Cas12a Assays

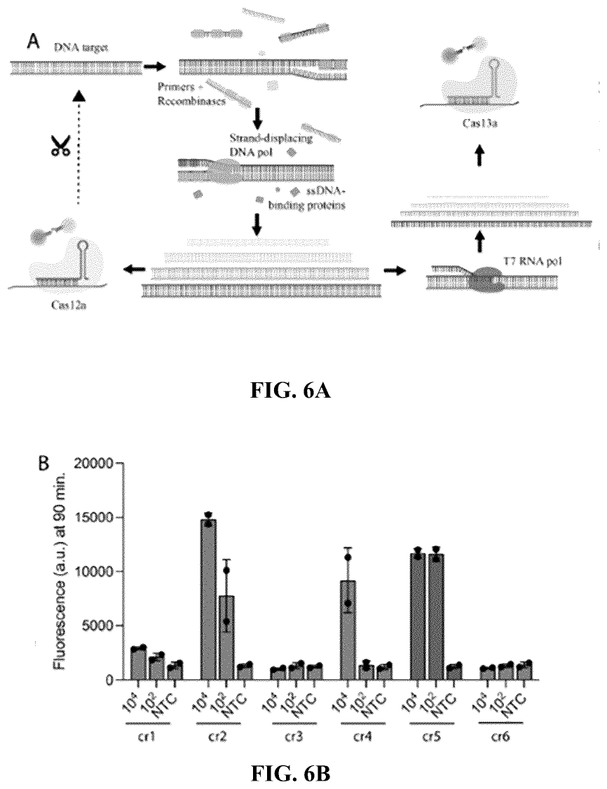

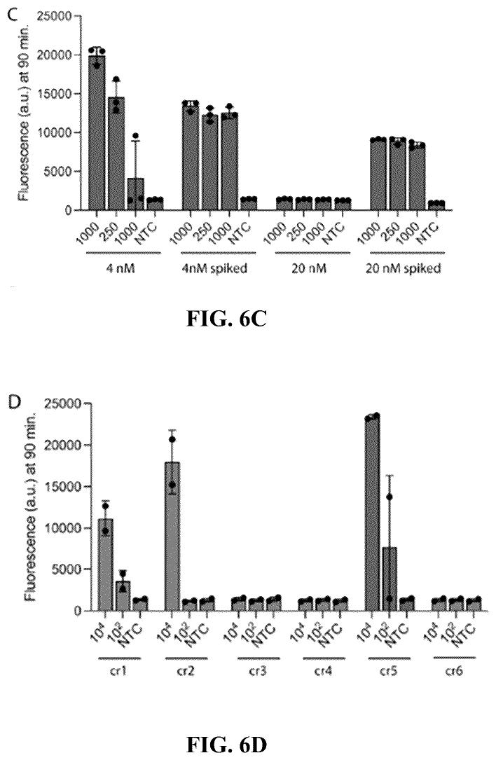

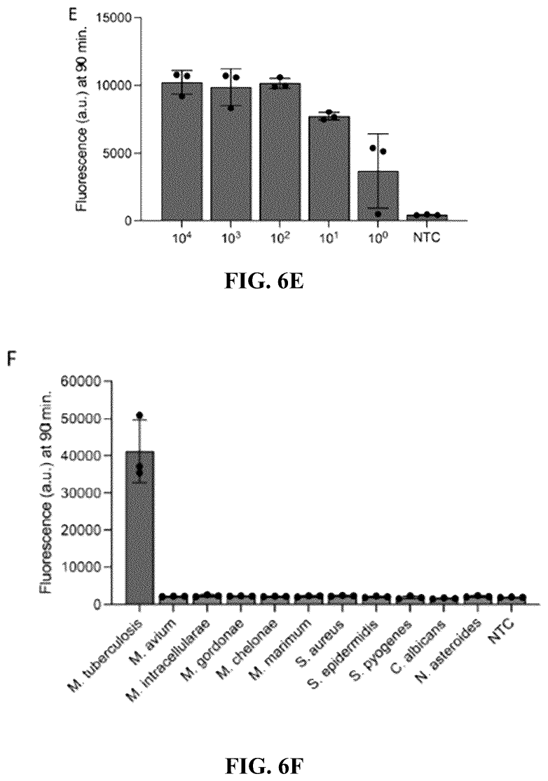

Two-step Cas12a assays were performed by mixing an amplification master mix (below) with target, allowing amplification to occur for 30 minutes before adding Cas12-crRNA RNPs (FIGS. 6B and 6C). Master mixes (concentrations given for final reaction) were generated by combining SHINE buffer (20 mM HEPES pH 8.0, 60 mM KCl, 3.5% PEG-8000, nuclease-free (nf) water), 1 U/μL RNAse Inhibitor (New England BioLabs), 70 nM of each RPA primer, 0.25 μM FAM 5C quenched reporter (IDT), and 14 nM MgOAc in nuclease-free (nf) water. TwistAmp® Basic RPA pellets were used for amplification, whereby 1 pellet was resuspended for every 107.5 μL of final reaction volume. The target (10% of final reaction volume) and amplification master mix were combined, lightly vortexed, and incubated at 37° C. for 30 minutes in an Agilent BioTek Cytation 5 microplate reader, taking fluorescence readings every 5 minutes (excitation: 485, emission 525). crRNAs and LbCas12a Ultra (10007922; IDT) in nf water at 10× their final equimolar concentration of 4 or 20 nM were combined on ice for 15-20 minutes and added to each reaction 30 minutes into the reaction. 15 μL reactions were performed in technical triplicates (with the exception of two early optimization experiments performed in duplicate to increase condition throughput) in 384-well clear-bottom microplates (788096; Greiner). Reactions were then returned to the plate reader at 37° C. for up to 3 hours of additional fluorescence readings.

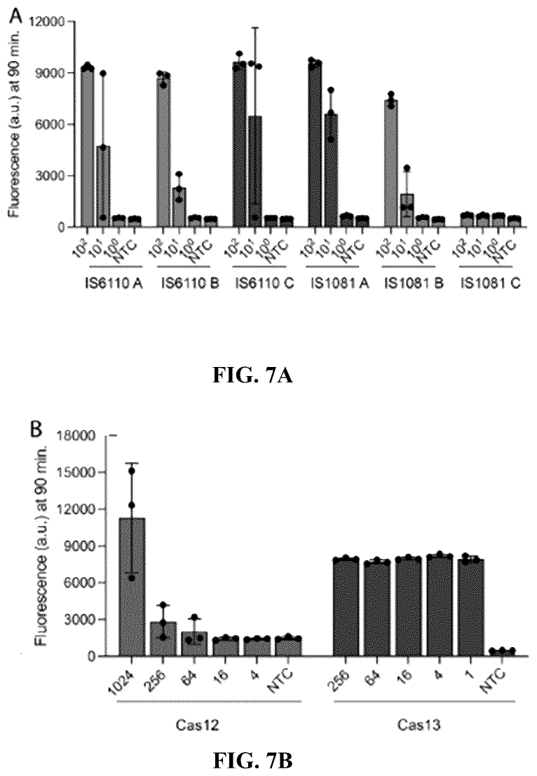

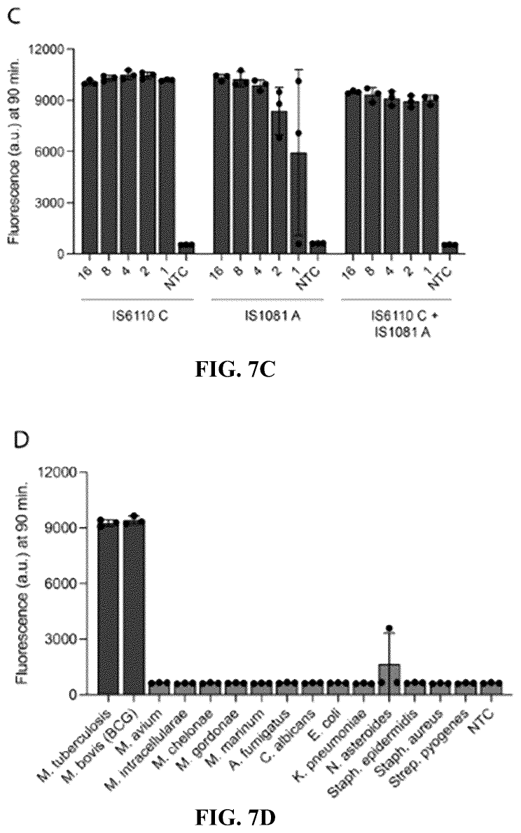

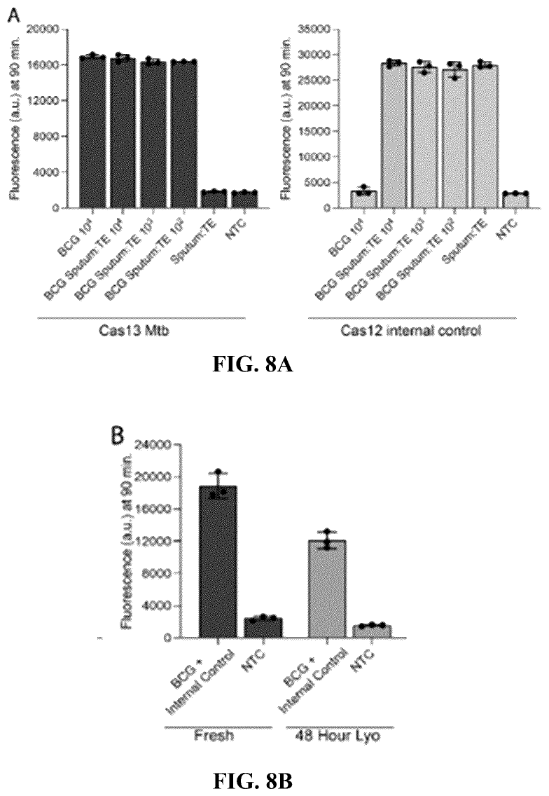

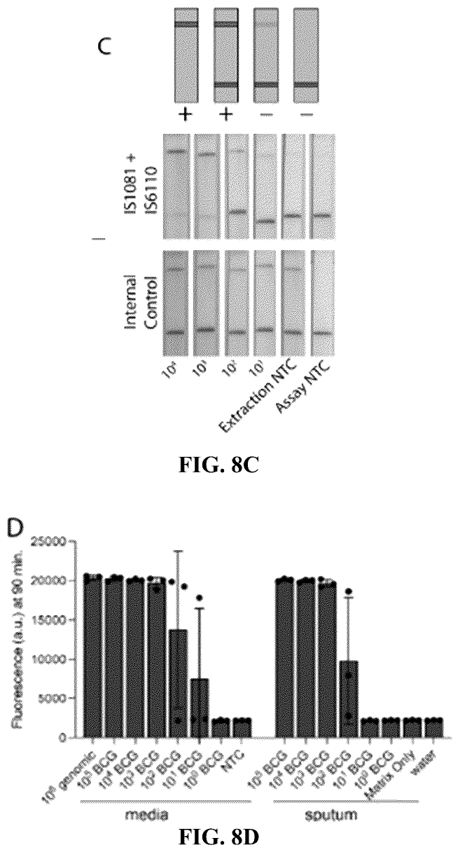

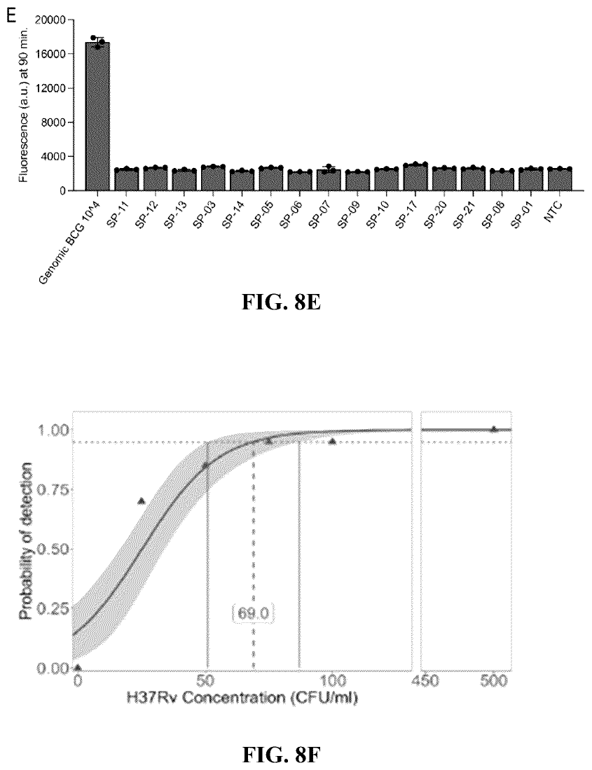

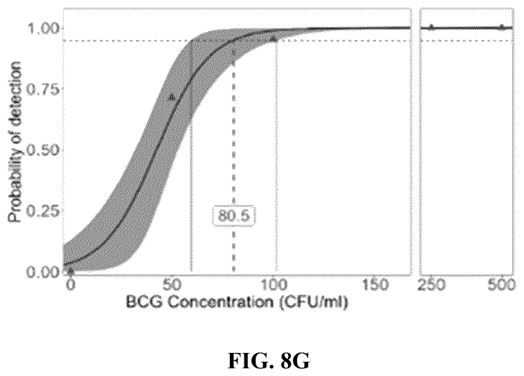

Single-step Cas12a assays were performed by mixing complete Cas12a master mix (90% by volume) with target (10% by volume) (FIGS. 6C, 6D, 6E, 6F, and 7D). Master mixes were prepared as in the two-step protocol, except crRNAs and Cas12a were added in the master mix before adding target, for a final concentration of 4 nM (FIGS. 6A-6F and 7A-7D), 30 nM (FIGS. 8A-8G), or as otherwise indicated. Corning 384-well microplates (CLS3544) were used for 15 μL reactions (FIGS. 6A-6F, 7A-7D and FIGS. 8A-8G). Reactions were incubated and measured as above.

### 5D. Single-Step Cas13a Assays

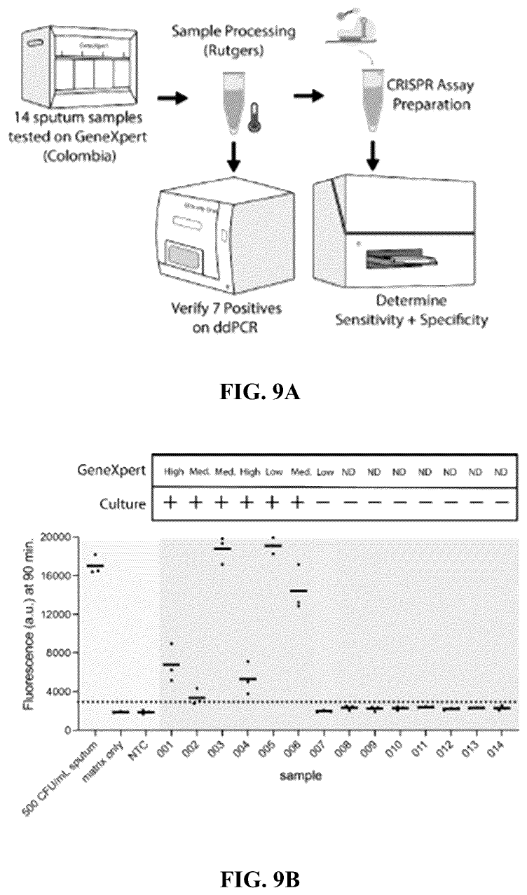

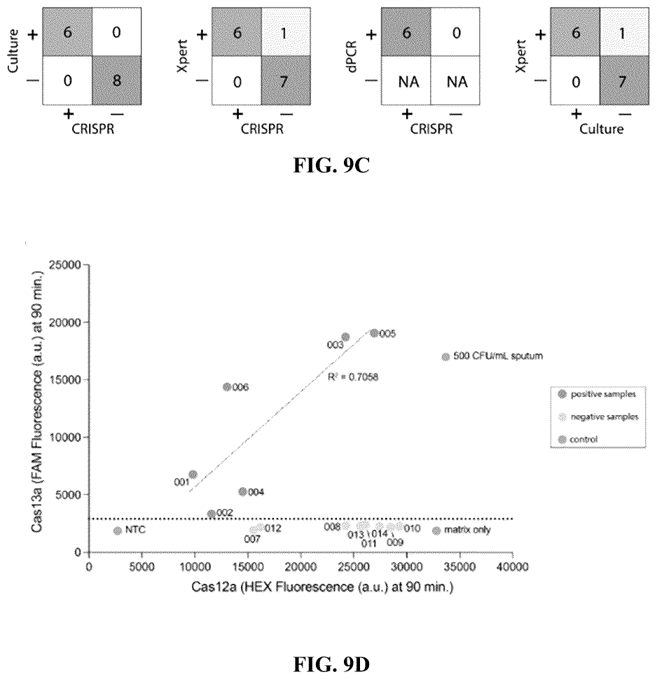

Single-step Cas13a assays were performed as for the single-step Cas12a reactions, except that to form the Cas13a master mix, the same SHINE buffer and 14 nM MgOAc were mixed with 45 nM LwaCas13a (Genscript, stored in 100 mM Tris HCl pH 7.5 and 1 mM DTT), 0.3 nM of each rNTP (New England Biolabs), 1 U/mL T7 RNAP (Biosearch Technologies), 62.5 nM FAM 6U quenched reporter (5′-/56-FAM/rUrUrUrUrUrU/3IABkFQ/-3′; IDT), 45 nM of a single crRNA (FIGS. 7A, 7B, and 7C) or 22.5 nM of each crRNA in a combination (FIGS. 7C, 7D, 8A-8G, and FIGS. 9A-9D), and 70 nM of the appropriate RPA primers in nf water. Reactions were loaded in technical triplicates into Greiner 384-well clear-bottom microplates for 15 μL reactions (FIG. 7D) or Corning 384-well microplates for 20 μL reactions (FIGS. 8A-8G and 9A-9D). The reaction was then incubated at 37° C. for up to 3 hours in a Cytation 5, as above. Single-step Cas13a assays were performed by mixing complete Cas12a master mix (90% by volume) with target (10% by volume). When target is sputum, pipette mix 10+ times and pulse vortex before spinning down.

Combined Cas12a/Cas13a assays were performed by combining all the non-redundant reagents from the two independent single-pot assays and replacing the DNA reporter with 0.25 M HEX 5C quenched reporter (IDT), to be measured in two fluorescence channels: excitation: 485, emission 525; excitation: 530, emission: 570.

### 5E. Lateral Flow Read-Outs

The assay was adapted to lateral flow with the following conditions: the quenched Cas13a reporter was replaced with 750 nM FAM 14U Bio (IDT) and the Cas12a reporter was replaced with FAM 5C Bio (IDT) when alone or FAM 5C Dig (IDT). Reactions were incubated in PCR strip tubes at 37° C. for 90 minutes before being diluted 1:4 in HybriDetect Assay buffer (Milenia). The reaction was equilibrated for 5 minutes before adding HybriDetect lateral flow strips for 1 minute. Images were taken 1 minute after dipstick removal.

### 5G. Lyophilization

Lyophilization methods were based on a previously described protocol (Arizti-Sanz et al. 2022), with some minor modifications. The dual assay was optimized to ensure integrity after lyophilization. The reaction was prepared as described above, except 20 mM HEPES (pH 8.0) was added in place of the complete SHINE buffer. Sucrose at 5% (w/v) and mannitol at 150 mM, were added as cryoprotectants. Assays were aliquoted (eight reactions per aliquot), flash frozen, and lyophilized at −30° C. for 24 hours using a Freezone Triad Benchtop Freeze Dryer from Labconco. Aliquots were then vacuum-sealed alongside a desiccant and stored at 4° C. until further use. Assays were resuspended in 3.5% PEG, 60 mM KCl, and 14 mM MgOAc in nf water, aliquoted, and mixed with target. Reactions were loaded in technical triplicates in Corning 384-well microplates for 20 μL reactions (FIGS. 8A-8G). Reactions were then incubated at 37° C. for up to 3 hours. Fluorescent measurements were taken every 5 minutes in a Cytation 5 microplate reader, as above.

### 5H. Collection of Clinical Samples

TB-negative samples for spiking were collected from patients of University Hospital, Newark New Jersey, USA, after written informed consent. Eligible participants were adults>18 years old diagnosed with a non-TB respiratory condition (e.g. cardiogenic pulmonary edema, asthma exacerbation, other bacterial pneumonia) and were willing and able to provide an expectorated sputum sample. Following written informed consent, symptomatic individuals undergoing evaluation for tuberculosis (TB) at two primary health centers in Cali, Colombia, provided expectorated sputum for examination. The study protocol was reviewed and approved by the Institutional Review Boards (IRBs) for ethical conduct in research involving human subjects at the Centro Internacional de Entrenamiento e Investigaciones Médicas (CIDEIM #1325). The protocol adhered to both national regulations (Resolution 008430, Ministry of Health, Republic of Colombia, 1993) and international ethical guidelines, including the Declaration of Helsinki and its amendments (World Medical Association, Fortaleza, Brazil, October 2013).

### 5I. Sample Processing

Sample processing was performed as described in the above Examples. Briefly, Chelex extraction for sputum samples: For individual patient screening, inhibitory patient screening, and dynamic range extractions. 100 μL of spiked sputum was aliquoted into a screw-cap Lysing Matrix B tube (MP Biomedicals, Cat. #MP116911100), subsequently 200 μL of Chelex-100 resin (instagene Matrix BIO-RAD, Cat. #7326030) was added. Samples were vortexed three times, for 30 seconds at 3400 RPM each time, subsequently incubated at 95° C. for 30 minutes, then centrifuged at 9400 RCF for 2 minutes. ~60-80 μL of supernatant were collected to serve as inputs for the diagnostic assays.

LoD Extractions: 400 μL of spiked sputum was aliquoted into a screw-cap Lysing Matrix B tube, subsequently 800 μL of Chelex-100 resin (instagene Matrix) was added. Samples were vortexed three times, for 30 seconds at 3400 RPM each time, subsequently incubated at 95° C. for 30 minutes, then centrifuged at 9400 RCF for 2 minutes. ~800 μL of supernatant was collected.

A convenience sample of symptomatic adults aged≥18 years and undergoing evaluation for tuberculosis (TB) at two primary health centers in Cali, Colombia, provided two expectorated or induced sputum samples on the same day. One was sent for smear microscopy and mycobacterial culture on liquid media (MGIT) on-site in the clinical laboratories, while the other was sent for molecular testing using the GeneXpert® MTB RIF Ultra assay at the CIDEIM research laboratory in accordance with the manufacturer's instructions.

### 5J. Clinical Sample Testing

Clinical samples were prepared using methods described above. Primers, crRNAs, and master mix are the same as above.

dPCR was performed in the QIAcuity® digital PCR system using the QIAcuity® 4× Probe PCR kit and 26 k 8-well nanoplate (QIAGEN). Previously published sequences for primers and probes were used (IS6110-I-F, IS6110-I-R, and IS6110-TM) (Chakravorty et al. 2017). For the final reaction volume of 12 μL, 3 μL of 4× Probe PCR Master mix, 0.8 μM concentration of forward and reverse primers, 0.4 μM concentration of probe, and 2 μL of DNA sample were added. Molecular grade, nuclease-free water was used to complete the volume. Thermal cycling conditions are: PCR initial heat activation at 95° C. for 2 minutes, followed by 40 cycles of denaturation at 95° C. for 15 seconds and combined annealing/extension at 60° C. for 30 seconds. DNA samples were tested both directly and at 10-fold dilution in replicates of three. M. bovis BCG DNA at 100 gc/reaction, and nuclease-free water were used as positive and negative controls, respectively.

5K. Data analysis

Plots and graphs were generated using Prism software.

Data were plotted in R using the ggplot2 package. Simple logistic regression was used to model detection probabilities for (a) H37RV and (b) BCG bacteria as a function of concentration (FIG. 8F). The 95% LOD was estimated as the concentration in which the probability of detection of a positive sample is 0.95. 95% Wald confidence intervals were generated from the standard error of the estimated concentrations.

Assay cutoffs for positive or negative results were derived using a 70:30 train-test split strategy. ROC curves were constructed from a dataset of 115 experiments with known bacterial concentrations. Four approaches were explored for setting the cutoff; (1) the mean of the negative samples plus three times the standard deviation of the negative samples, (2) the point on the ROC curve corresponding to the maximum Youden index, (3) the point on the ROC curve corresponding to the WHO 2024 Target Product Profile (TPP) sensitivity standard for a sputum-based near-point-of-care test and (4) the point on the ROC curve corresponding to the WHO 2024 specificity standard for all diagnostic tests (Table 7). Both the Youden index and the specificity standard approaches gave a cutoff of 2895.42 fluorescent units, which was selected and rounded to 2900 for the final cutoff.

### Example 6. One-Pot Cas12a Assay Accurately Detects Mtb at Genome-Level Sensitivity

A streamlined Cas12a-based diagnostic workflow for Mtb was explored, where isothermal amplification of the DNA target using RPA is paired with direct detection by Lachnospiraceae bacterium Cas12a (LbCas12a, referred to herein as Cas12a). A Cas12a workflow is simpler than a Cas13a workflow because the enzyme directly detects the RPA product, eliminating the need for in vitro transcription (FIG. 6A). A set of six primer and Cas12a guide sets were designed and tested, targeting the multicopy insertion sequences IS6110 and IS1081, found uniquely among Mtb and related TB-causing Mtb complex (MTBC) bacteria (see Methods described herein).

To compare the efficiencies of each design for amplification and detection as isolated events, RPA was performed for 30 minutes before spiking Cas12a into the reactions (FIG. 6B). Primer-guide designs cr2 and cr5 outperformed other assays, detecting 100 copies of the synthetic insertion sequence per μL of the input synthetic DNA (FIG. 6B). As cis cleavage of the target amplicon in a one-pot reaction interferes with further amplification, this study aimed to maximize the potential for RPA to overcome the rate of Cas12a cis cleavage. The concentration of Cas12a was varied in one-pot and spiked-enzyme conditions targeting synthetic copies of the insertion sequence, finding that lower concentrations of Cas12a improved the sensitivity of the one-pot assay (FIG. 6C). Design cr5 appeared to be the most effective combination of primers and a guide in these improved conditions for one-pot detection targeting the synthetic insertion sequence (FIG. 6D). Even with conditions modified to improve assay sensitivity, the one-pot format still performed less sensitively than did the initial spike-in experiments, as seen for designs cr2, cr4, and cr5 (FIGS. 6B and 6D).

Having optimized the Cas12a assay on synthetic targets, its performance was tested against extracted genomic DNA from the Mtb reference strain, H37Rv, and extracts from nontuberculous mycobacteria (NTMs) and other pathogens. IS6110 appears in the H37Rv genome 17 times, and despite the added complexity of the genomic material as compared to synthetic copies of the insertion sequence, the one-pot Cas12a cr5 assay could detect H37Rv DNA at 10 genomes/μL (~170 copies/μL of IS6110) and even appeared to detect down to 1 genome/μL in 2 out of 3 technical replicates, approaching the probabilistic limits of genome loading in a reaction containing 1.5 μL of sample (FIG. 6E). Design cr5 was further tested against ten different NTMs and other pathogens and showed no cross-reactivity when 1 ng/μL of each genome was used as input (FIG. 6F).

### Example 7. Cas13a Assay for Mtb Detection Overcomes Cas12a Limitations

The decreased sensitivity of the Cas12a-based assays in a one-pot format seen in FIG. 6D compared to FIG. 6B was due to Cas12a cleaving the target DNA in cis before sufficient amplification could occur, thereby reducing exponential target amplification and trans cleavage of the reporter (FIG. 6A). As Cas13a requires an RNA target and cleaves RNA in trans, this study sought to determine if Cas13a provides higher sensitivity in a single pot reaction as it would not disrupt amplification in the same manner as Cas12a despite requiring in vitro transcription.

To test this, an Activity-informed Design with All-inclusive Patrolling of Targets (ADAPT) was used to design six different guide sets for Leptotrichia wadei (Lwa) Cas13a (herein referred to as Cas13a) for two high copy number insertion sequences, three targeting IS6110 and three targeting IS1081 (Metsky et al. 2022). Each of these designs were tested experimentally using synthetic DNA targets in a one-pot format. Two designs showed promise, IS6110c and IS1081a, as they both detected 2 out of 3 technical replicates of 10 copies/μL of their respective synthetic DNA targets, and all higher target concentrations (FIG. 7A).

Next, a direct comparison of Cas12a and Cas13a detection for IS6110 was performed. The amplicons for the Cas12a cr5 assay and the Cas13a IS6110c assay overlap directly, as do the protospacers of their guide RNAs. Both the Cas12a- and Cas13a-based one-pot assays were tested head-to-head using a dilution series of synthetic IS6110 DNA targets. Cas13a IS6110c was able to pick up 1 copy/μL of IS6110, whereas Cas12a cr5 began to lose sensitivity at 384 copies/μL (FIG. 7B). This supports the higher sensitivity of Cas13a compared to Cas12a when amplification is combined with detection without including a temporal barrier to Cas12a activity. In subsequent experiments, Cas13a was used as the preferred detection enzyme for Mtb.

As different MTBCs have variable copy numbers of insertion sequences, this study sought to simultaneously detect both IS6110 and IS1081 in a dual detection assay. IS1081a was less sensitive than IS6110c when tested against the H37Rv genome, as could be expected for this genome carrying 17 full copies of IS6110 and 5 full, 1 partial copies of IS1081 (Chitale et al. 2022) (FIG. 7C). Combining IS1081a and IS6110c together did not appear to hamper either assay's sensitivity when testing against the H37Rv genome (FIG. 7C). The specificity of the combined IS6110 and IS1081 dual detection assay was further tested against 13 NTMs and other pathogens, alongside two positive controls, H37Rv and Bacille Calmette-Guerin (BCG), an MTBC that harbors a single copy of IS6110. No NTM or other tested pathogen significantly triggered off-target signal for the dual detection assay at 0.1 ng of the respective genome per L of sample (FIG. 7D).

### Example 8. Adapting Combined CRISPR Assays to Address Clinical Needs

As Mtb can prove difficult to lyse due to its cell wall, a positive internal control was incorporated into the workflow to mitigate false negatives caused by inhibitors, salivary dilution, or poor nucleic acid extraction (Amaro et al. 2008). After evaluating several targets, a Cas12a-based internal control was included that targets the Long Terminal Repeat (LTR) of the endogenous retrovirus ERVK, as each human chromosome carries between 40 and 400 copies of the element (Lander et al. 2001). Due to the target's abundance in human DNA, detection was possible without reducing the concentration of Cas12a for the optimized one-pot assay, even for challenging sample types.

SHINE is compatible with sputum, the gold-standard sample type for active TB diagnosis. In order to circumvent the need for specialized equipment and to keep hands-on time minimal, a novel sample processing method was used, which was developed in parallel with this assay to prepare sputum samples for detection by the CRISPR assays and other nucleic acid amplification methods (see methods). Sputum is an inherently variable biofluid whose macroscopic properties (viscosity, color, etc.) can vary over time even for an individual patient. When testing individual sputum samples, it was initially observed that a small subset of samples sporadically inhibited all tested RPA-CRISPR assays. However, much of this inhibitory effect could be eliminated by diluting the samples 1:1 with Tris-EDTA (TE) buffer, allowing for reliable detection of both Mtb and human DNA using Cas13a and Cas12a in all sputum samples, despite a minimal reduction in assay sensitivity (FIG. 8A). Hereafter, all references to sputum refer to sputum samples diluted 1:1 with TE buffer. The dynamic range of the assay was tested by spiking BCG genomic DNA at known concentrations into pooled and diluted sputum samples collected from TB-negative patients at University Hospital in Newark, NJ. As low as 10² colony-forming units (CFU)/mL BCG in pooled samples (representing between 0 and 5 copies of IS6110 per assay, based on later digital PCRs (dPCRs); Tables 3-5) were consistently detected by both the Cas12a internal control and Cas13a dual detection assay, indicating compatibility with this clinically relevant sample type (FIG. 8A).

For ease of use, Cas12a and the dual detection Cas13a assay were combined in a single SHINE reaction. However, this integration greatly affected sensitivity and slowed the rate of signal accumulation for both enzymes. While screening potential improvements to this combined Cas12a and Cas13a assay, it was determined that excluding any one of the three amplification-detection sets restored this sensitivity, likely the result of competing exponential demands on shared RPA machinery (FIGS. 7A-7D). As sensitivity and sample coverage are critical to the utility of any diagnostic assay, the Cas12a internal control and Cas13a dual detection assay were split into two chambers, one with the internal control and one dedicated to Mtb detection. As these assays are performed in parallel, the separation of components should minimally increase labor; herein, this parallelized assay format is referred to as SHINE-TB.

As TB affects primarily underserved areas, it is important to have an accessible and field deployable assay format. Similar molecular diagnostics have previously been shown to be amenable to freeze-drying with minimal effects on assay performance. Single-use tubes of SHINE-TB were lyophilized for 24 hours and stored at 4° C. for 48 hours before testing the resuspended assays on BCG-spiked sputum, yielding comparable results to their fresh assay counterparts, albeit with a reduction to maximum fluorescence in this fluorometric assay format (FIG. 8B). The reporter was also converted to be compatible with a lateral flow readout, whereby the quencher at one end of the reporter is replaced by biotin; intact reporters are immobilized at a lower streptavidin band while the antigenic ends of cleaved reporters freely migrate past to reach the upper band (disappearance of the lower biotin line is not required; faint positive test lines are interpreted as negative; FIG. 8C). Despite these modifications, the assay consistently detected the internal control and 10² CFU/mL of BCG like its fluorometric counterpart.

### Example 9. Characterizing Assay Performance in Contrived Clinical Samples

To further characterize the finalized assay, fresh, fluorometric SHINE-TB was moved to pooled sputum samples spiked with BCG at various concentrations prior to sample processing. As a control for the sample matrix, BCG cultures in culture media were also tested. It was found that sputum did not change the upper limit of detection but did affect the lower limit of the Cas13a dual detection assay, in that samples at 10² CFU/mL (representing 0-9 copies/μL of IS6110) appeared to be near the threshold of detection (FIG. 8D, Tables 3 and 4). Changes to the abundance of BCG did not affect internal control signals.

The specificity of fresh SHINE-TB was tested against individual sputum samples from fourteen TB-negative patients to mimic real-world sample variability. Sputum samples were collected from patients displaying TB-like symptoms, but whose samples were confirmed Mtb negative using dPCR (FIG. 8E). The Mtb-negative samples did not elicit any false positive assay detections, although two samples, SP-11 and SP-09, were not detected by the internal control, suggesting variable sample extraction or assay inhibition. Of note, SP-10 was a separate sputum sample collected from the same donor as SP-09, which did not inhibit the assay, showing that there is variability even in samples collected from the same patient.

To determine the limit of detection (LoD) of the Cas13a dual detection assay, various concentrations were screened of BCG (750 CFU/mL to 50 CFU/mL) or H37Rv (500 CFU/mL to 25 CFU/mL) spiked into pooled sputum before sample processing. LoD fluorescence cutoffs were established as the average fluorescence of the sputum-only controls plus three times their standard deviation (SD). The LoDs were thus the concentrations at which this threshold was reached, as inferred by a logistic regression curve. The LoD for BCG was 80.5 (59.4-101.6) CFU/mL (FIG. 8F). All concentrations above 100 CFU/mL were detected and 2/3 of the 50 CFU/mL samples were detected. At 50 CFU/mL, there is ~1 copy/μL of IS6110 in the input sample, ~2 copies per L reaction, according to dPCR (Table 5). Note, due to the low copy number and high viscosity of sputum, samples containing below 500 CFU/mL required mixing by pipetting 10+ times. All samples were positive for the internal control. The LoD for H37Rv was 69.0 (51.0-86.9) CFU/mL (FIG. 8G). As expected, all samples were detected by the internal control assay. Overall, SHINE-TB performed sensitively in clinically relevant sample types.

### Example 10. SHINE-TB Performs Well on Patient Samples

SHINE-TB was blind-tested on fourteen randomized patient samples that were previously diagnosed using GeneXpert® MTB/RIF (7 Mtb-positive, 7 Mtb-negative). First, the samples were processed using the two-step extraction method before SHINE-TB and dPCR (FIG. 9A). 6 samples were identified as positive and 8 samples as negative (FIG. 9B and Table 7). This agrees with results from GeneXpert® MTB/RIF except for sample 007, resulting in 86% sensitivity and 100% specificity compared to GeneXpert® MTB/RIF (FIG. 9C). GeneXpert® MTB/RIF was tested, which confirmed positive samples on dPCR and found 6 out of 7 samples to be positive (FIG. 9C and Table 6). Culture results also matched SHINE-TB results, finding 6 patients positive and 8 negative. GeneXpert® MTB/RIF falsely identified sample 007 as positive and differences in results are not due to sample processing or degradation (FIGS. 9B and 9C, and Table 6). For further information on the samples, see Table 6.

The signal intensity for the MTBC vs internal control assays was also plotted (FIG. 9D). For the samples with positive MTBC signal, there was a positive correlation between Cas13a signal and Cas12a signal, supporting that internal control performance reflects the performance of the MTBC assay and thus may help indicate issues with sample extraction or inhibition.

This study demonstrated the accuracy and feasibility of SHINE-TB for the detection of MTBC DNA and human DNA. Two separate Cas12a- and Cas13a-based assays for the detection of MTBC were developed and compared, finding that Cas13a performs better for the purposes of single-pot, manipulation-free diagnostic assays. These assays were designed to create a one-step platform that does not require a mechanical separation between amplification and detection. Despite integrating suboptimal PAM sequences to attempt to combat the competition between Cas12a and amplification, it was found that Cas13a had greater sensitivity than Cas12a, demonstrating the benefits of a workflow whose amplification substrates are not also degraded enzymatically as part of detection despite the added complexity of longer primers to initiate transcription and additional substrates needed for T7 RNA polymerase. Others have favored Cas13 despite this added complexity, possibly due to its higher turnover for trans-cleavage.

A Cas13a dual detection assay for Mtb to accommodate a larger variety of tuberculosis-causing MTBCs, as various species have varying insertion copy numbers. Although Cas13 was favored for a one-pot test that required sensitivity and robustness, Cas12a proved suitable for an internal control targeting a highly abundant genetic element in properly extracted human samples.

This study demonstrated the feasibility of lyophilization and lateral flow readout to provide a means for deployability to lower resource areas. If the two were combined, and considering the few step sample preparation, this would allow for minimal liquid handling and minimal equipment required. Entirely equipment-free SHINE is possible as body heat is the optimal temperature for both RPA and Cas13a. Previous CRISPR-based TB studies have successfully incorporated lateral flow, but not in a one-pot format. This is the first TB test where amplification and detection are combined that is compatible with both lateral flow and lyophilization.

SHINE-TB is also compatible with sputum. Compared to GeneXpert® MTB/RIF Ultra, significantly less sample volume is used, only using 100 μL per extraction and ~2 μL of diluted sputum per reaction to their ~1-3 mL (Helb et al. 2010). Despite contributing just 5% of the overall reaction volume, some individual sputum types showed significant assay-inhibition for both Cas13a and Cas12a. A thorough purification or extraction step is not performed before testing, so it is possible these samples contain inhibitors to RPA, as has been seen for PCR. Due to the internal control, these false negatives will be detected and allow these samples to be labeled as indeterminate, preventing misdiagnosis.

The assay was coupled with a simple sample extraction process to see whether the assay has a limit of detection that is adequate for clinical samples. Using sputum as an input, an LoD of ~80 CFU/mL and ~60 CFU/mL for BCG and H37Rv, respectively, was identified with both targets being detected nominally throughout a wide dynamic range of concentrations. The LoD for H37Rv was higher than expected, considering the increased copy number of targets for H37Rv compared to BCG. As dPCR testing of 50 CFU/mL BCG found ~1 copy/μL of IS6110, which would correspond to about one genome per microliter, it is possible at these low concentrations that some reactions do not contain any target genomes (Table 5). Thus, at these concentrations, increased copy number does not significantly increase detection. Regardless, the clinical sample testing established that SHINE can detect Mtb in clinical samples with 10000 specificity and 100% o sensitivity compared to culture.

This simple one-step assay provides robust detection of both MTBC and human DNA, enabling accessible and reliable detection of this deadly disease.

TABLE 3
dPCR data on sputum dilutions for Dynamic Range experiment 3D.
(Sputum + BCG): TE IS6110 copies/μl

| CFU/ | | | | | | |
|---|---|---|---|---|---|---|
| ml | rep 1 | | rep 2 | | rep 3 | |
| 10⁵ | 844.8 | 834.6 | 1048.8 | 1111.2 | 652.2 | 636.6 |
| 10⁴ | 100.2 | 66.12 | 55.572 | 74.7 | 155.28 | 143.52 |
| 10³ | 11.334 | 18.522 | 14.196 | 9.348 | 14.292 | 14.496 |
| 10² | 4.548 | 0 | 9.432 | N/A | 4.764 | 0 |
| 10¹ | 0 | 0 | 0 | 2.382 | 0 | 0 |
| 10⁰ | 0 | 0 | 0 | 0 | 0 | 0 |
| 0 | 0 | 0 | 0 | 0 | 0 | 0 |
| NTC | 0 | 0 | | | | |

TABLE 4
dPCR data on media dilutions for Dynamic Range experiment 3D.
(media + BCG): TE IS6110 copies/μl

| CFU/ | | | | | | |
|---|---|---|---|---|---|---|
| ml | rep 1 | | rep 2 | | rep 3 | |
| 10⁵ | 1881.6 | 1873.2 | 2004 | 2146.2 | 1392 | 1381.2 |
| 10⁴ | 249.18 | 264.72 | 119.34 | 112.68 | 81.42 | 129.48 |
| 10³ | 14.37 | 7.182 | 18.708 | N/A | 23.028 | 15.978 |
| 10² | 0 | 2.406 | 2.424 | 0 | 5.028 | 0 |
| 10¹ | 0 | 0 | 0 | 2.4 | 0 | 0 |
| 10⁰ | 0 | 0 | 0 | 0 | 0 | 0 |
| 0 | 0 | 0 | 0 | 0 | 0 | 0 |
| NTC | 0 | 0 | | | | |

TABLE 5
dPCR results for BCG LoD.

| | Sputum: | Media: | |
|---|---|---|---|
| | TE IS6110 | TE IS6110 | |
| CFU/mL | copy # per μL | copy # per μL | Control |
| 1000 | 27.2 | 33.46 | NA |
| 750 | 28.44 | 43.12 | NA |
| 500 | 12.78 | 13.16 | NA |
| 250 | 9.64 | 11.1 | NA |
| 100 | 3.22 | 4.42 | NA |
| 50 | 1.08 | 1.1 | NA |
| Negative | 0 | 0 | NA |
| Control | | | |
| H37Ra | NA | NA | 44238 |
| gDNA 10³ | | | |
| copies | | | |
| NTC | NA | NA | 0 |

TABLE 6
Patient sample information for FIGS. 9A-9D.

| | | | Xpert Ct | dPCR | |
|---|---|---|---|---|---|
| | | | Value | copies/2 μL | |
| | | Xpert | (Colombia) | (Rutgers) | |
| # | Patient # | (Colombia) | IS-1081/6110 | IS-1081/6110 | Description |
| 001 | TB-02-0019 | Medium | 16.2 | 10890.06667 | Smear (+), Culture (+), |
| | | | | | GeneXpert sputum (+) |
| 002 | TB-01-0083 | High | 15.9 | 16065.7 | Smear (+), Culture (+), |
| | | | | | GeneXpertsputum (+) |
| 003 | TB-01-0111 | Medium | 15.8 | 358.6633333 | Smear (+), Culture (+), |
| | | | | | GeneXpertsputum (+) |
| 004 | TB-01-0112 | High | 15.9 | 37198 | Smear (+), Culture (+), |
| | | | | | GeneXpertsputum (+) |
| 005 | TB-01-0120 | Low | 16.5 | 34 | Smear (+), Culture (+), |
| | | | | | GeneXpertsputum (+) |
| 006 | TB-01-0159 | Medium | 16 | 3453.76 | Smear (+), Culture (+), |
| | | | | | GeneXpertsputum (+) |
| 007 | TB-01-0162 | Low | 20.2 | 0 | Smear (ND), Culture (−), |
| | | | | | GeneXpertsputum (+) |
| 008 | TB-02-0018 | Not Detected | 0 | NA | Smear (−), Culture (C), |
| | | | | | GeneXpertsputum (−) |
| 009 | TB-02-0020 | Not Detected | 0 | NA | Smear (−), Culture (−), |
| | | | | | GeneXpertsputum (−) |
| 010 | TB-02-0021 | Not Detected | 0 | NA | Smear (−), Culture (−), |
| | | | | | GeneXpertsputum (−) |
| 011 | TB-02-0022 | Not Detected | 0 | NA | Smear (−), Culture (−), |
| | | | | | GeneXpertsputum (−) |
| 012 | TB-02-0023 | Not Detected | 0 | NA | Smear (ND), Culture (−), |
| | | | | | GeneXpertsputum (−) |
| 013 | TB-01-0137 | Not Detected | 0 | NA | Smear (−), Culture (−), |
| | | | | | GeneXpertsputum (−) |
| 014 | TB-01-0145 | Not Detected | 0 | NA | Smear (−), Culture (−), |
| | | | | | GeneXpertsputum (−) |

TABLE 7
Fluorescence cutoff point derivation and
associated sensitivities and specificities.

| | | Cutoff | | |
|---|---|---|---|---|
| | | Value | Sensitivity | Specificity |
| | | (FU) | (95% CI) | (95% CI) |
| Mean + 3 SD of | | | | |
| negative controls | | | | |
| Training set | (n = 83) | 3287.03 | 0.93 (0.85, 1.00) | 1.00 (1.00, 1.00) |
| Test set | (n = 32) | | 1.00 (1.00, 1.00) | 1.00 (1.00, 1.00) |
| Youden index | | | | |
| Training set | (n = 83) | 2895.42 | 0.96 (0.89, 1.00) | 1.00 (1.00, 1.00) |
| Test set | (n = 32) | | 0.93 (0.80, 1.00) | 1.00 (1.00, 1.00) |
| TPP 2024 | | | | |
| sensitivity standard* | | | | |
| Training set | (n = 83) | 7047.50 | 0.85 | 1.00 (1.00, 1.00) |
| Test set | (n = 32) | | 0.82 (0.65, 1.00) | 1.00 (1.00, 1.00) |
| TPP 2024 | | | | |
| specificity standard | | | | |
| Training set | (n = 83) | 2895.42 | 0.96 (0.89, 1.00) | 0.98 |
| Test set | (n = 32) | | 0.93 (0.80, 1.00) | 1.00 (1.00, 1.00) |

TABLE 8
Sequences used in Examples 5-10

| | Oligo type; | | |
|---|---|---|---|
| Name | company | Sequence | |
| | | | Gene assay |
| | | | name |
| IS6110_cr1 | custom | /AltR1/rUrArArUrUrUrCrUrArCrUrArArGrU | Cas12_IS6110_ |
| | RNA; IDT | rGrUrArGrArUrCrUrArCrCrCrArCrArGrCrCr | cr1 |
| | | GrGrUrUrArGrGrU/AltR2/ (SEQ ID NO: 10) | |
| IS6110_cr2 | custom | /AltR1/rUrArArUrUrUrCrUrArCrUrArArGrU | Cas12_IS6110_ |
| | RNA; IDT | rGrUrArGrArUrArCrCrGrGrCrUrGrUrGrGrGr | cr2 |
| | | UrArGrCrArGrArC/AltR2/ (SEQ ID NO: 11) | |
| IS6110_cr3 | custom | /AltR1/rUrArArUrUrUrCrUrArCrUrArArGrU | Cas12_IS6110_ |
| | RNA; IDT | rGrUrArGrArUrArArArGrArCrCrGrCrGrUrCr | cr3 |
| | | GrGrCrUrUrUrCrU/AltR2/ (SEQ ID NO: 12) | |
| IS6110_cr4 | custom | /AltR1/rUrArArUrUrUrCrUrArCrUrArArGrU | Cas12_IS6110_ |
| | RNA; IDT | rGrUrArGrArUrCrCrGrCrGrGrGrUrGrGrUrCr | cr4 |
| | | CrCrGrGrArCrArG/AltR2/ (SEQ ID NO: 13) | |
| IS6110_cr5 | custom | /AltR1/rUrArArUrUrUrCrUrArCrUrArArGrU | Cas12_IS6110_ |
| | RNA; IDT | rGrUrArGrArUrCrGrGrGrCrArCrCrGrUrArAr | cr5 |
| | | ArCrArCrCrGrUrA/AltR2/ (SEQ ID NO: 14) | |
| IS6110_cr6 | custom | /AltR1/rUrArArUrUrUrCrUrArCrUrArArGrU | Cas12_IS6110_ |
| | RNA; IDT | rGrUrArGrArUrArArArUrCrGrCrGrUrUrCrGr | cr6 |
| | | CrCrCrUrUrCrGrC/AltR2/ (SEQ ID NO: 15) | |
| IS1081_cr1 | custom | /AltR1/rUrArArUrUrUrCrUrArCrUrArArGrU | Cas12_IS1081_ |
| | RNA; IDT | rGrUrArGrArUrGrCrGrCrCrArGrArUrCrUrGr | cr1 |
| | | CrUrUrGrGrGrGrA/AltR2/ (SEQ ID NO: 16) | |
| IS1081_cr3 | custom | /AltR1/rUrArArUrUrUrCrUrArCrUrArArGrU | Cas12_IS1081_ |
| | RNA; IDT | rGrUrArGrArUrGrCrCrArUrGrArUrCrGrArCr | cr3 |
| | | ArCrUrUrGrCrGrA/AltR2/ (SEQ ID NO: 17) | |
| IS1081_cr4 | custom | /AltR1/rUrArArUrUrUrCrUrArCrUrArArGrU | Cas12_IS1081_ |
| | RNA; IDT | rGrUrArGrArUrUrCrArCrArCrCrArArGrUrGr | cr4 |
| | | UrUrUrCrGrArCrC/AltR2/ (SEQ ID NO: 18) | |
| C13_IS6110_A | custom | /AltR1/rGrArUrUrUrArGrArCrUrArCrCrCrC | Cas13_IS6110_ |
| | RNA; IDT | rArArArArArCrGrArArGrGrGrGrArCrUrAr | A |
| | | ArArArCrGrGrCrArUrCrGrArGrGrUrGrGrCr | |
| | | CrArGrArUrGrCrArCrCrGrUrCrG/AltR2/ | |
| | | (SEQ ID NO: 19) | |
| C13_IS6110_B | custom | /AltR1/rGrArUrUrUrArGrArCrUrArCrCrCrC | Cas13_IS6110_ |
| | RNA; IDT | rArArArArArCrGrArArGrGrGrGrArCrUrAr | B |
| | | ArArArCrGrArCrGrArUrCrArArCrGrGrCrCr | |
| | | UrArUrArCrArArGrArCrCrGrArG/AltR2/ | |
| | | (SEQ ID NO: 20) | |
| C13_IS6110_sg5 | custom | /AltR1/rGrArUrUrUrArGrArCrUrArCrCrCrC | Cas13_IS6110_ |
| | RNA; IDT | rArArArArArCrGrArArGrGrGrGrArCrUrAr | C |
| | | ArArArCrCrUrArCrGrGrUrGrUrUrUrArCrGr | |
| | | GrUrGrCrCrCrGrCrArArArGrUrG/AltR2/ | |
| | | (SEQ ID NO: 21) | |
| C13_IS1081_A | custom | /AltR1/rGrArUrUrUrArGrArCrUrArCrCrCrC | Cas13_IS1081_ |
| | RNA; IDT | rArArArArArCrGrArArGrGrGrGrArCrUrAr | A |
| | | ArArArCrCrArArArGrCrUrUrUrCrCrArArGr | |
| | | UrCrGrCrArArGrUrGrUrCrGrArU/AltR2/ | |
| | | (SEQ ID NO: 22) | |
| C13_IS1081_B | custom | /AltR1/rGrArUrUrUrArGrArCrUrArCrCrCrC | Cas13_IS1081_ |
| | RNA; IDT | rArArArArArCrGrArArGrGrGrGrArCrUrAr | B |
| | | ArArArCrGrCrArGrCrGrCrCrGrCrArArGrCr | |
| | | GrArGrCrUrGrArArCrGrCrGrCrA/AltR2/ | |
| | | (SEQ ID NO: 23) | |
| C13_IS1081_C | custom | /AltR1/rGrArUrUrUrArGrArCrUrArCrCrCrC | Cas13_IS1081_ |
| | RNA; IDT | rArArArArArCrGrArArGrGrGrGrArCrUrAr | C |
| | | ArArArCrGrCrGrGrGrCrUrArCrCrGrCrGrAr | |
| | | ArCrGrCrArGrCrGrArUrGrArGrC/AltR2/ | |
| | | (SEQ ID NO: 24) | |
| LR5_crRNA_1 | custom | /AlTR1/rUrArArUrUrUrCrUrArCrUrArArGr | Cas12_ |
| | RNA; IDT | UrGrUrArGrArUrArCrArArArGrCrArCrArU | internalControl_ |
| | | rCrUrUrGrCrArCrCrGrC/AlTR2/ (SEQ ID | 1 |
| | | NO: 25) | |
| LR5_crRNA_2 | custom | /AlTR1/rUrArArUrUrUrCrUrArCrUrArArGr | Cas12_ |
| | RNA; IDT | UrGrUrArGrArUrArArGrGrArGrCrArUrGrC | internalControl_ |
| | | rUrGrCrCrUrUrCrArArG/AlTR2/ (SEQ ID | 2 |
| | | NO: 26) | |
| LR5_crRNA_3 | custom | /AltR1/rUrArArUrUrUrCrUrArCrUrArArGrU | Cas12_ |
| | RNA; IDT | rGrUrArGrArUrArArArGrCrArCrArGrCrArC | internalControl_ |
| | | rUrUrArArUrCrCrU/AltR2/ (SEQ ID NO: 27) | 3 |
| IS6110_cr1-2_F | custom | CGTAGGCGAACCCTGCCCAGGTCGACA | Cas12_IS6110_cr1 & |
| | DNA; IDT | CATAGG (SEQ ID NO: 28) | Cas12_IS6110_cr2 |
| IS6110_cr1-2_R | custom | CGATCTCGTCCAGCGCCGCTTCGGACC | Cas12_IS6110_cr1 & |
| | DNA; IDT | ACCA (SEQ ID NO: 29) | Cas12_IS6110_cr2 |
| IS6110_cr3_F | custom | AATTAGCGTGCTGGCCGGTCGAGCTCG | Cas12_IS6110_cr3 |
| | DNA; IDT | GCC (SEQ ID NO: 30) | |
| IS6110_cr3_R | custom | GGGACAACGCCGAATTGCGAAGGGCG | Cas12_IS6110_cr3 |
| | DNA; IDT | AACG (SEQ ID NO: 31) | |
| IS6110_cr4_F | custom | TGTGGCCGGATCAGCGATCGTGGTCCT | Cas12_IS6110_cr4 |
| | DNA; IDT | GCGGGCTT (SEQ ID NO: 32) | |
| IS6110_cr4_R | custom | AGATGCACCGTCGAACGGCTGATGACC | Cas12_IS6110_cr4 |
| | DNA; IDT | AAACTCGG (SEQ ID NO: 33) | |
| IS6110_cr5_F | custom | TGGCCACCTCGATGCCCTCACGGTTCA | Cas12_IS6110_cr5 |
| | DNA; IDT | GGGTTAGCCACAC (SEQ ID NO: 34) | |
| IS6110_cr5_R | custom | GCGAACTCAAGGAGCACATCAGCCGCG | Cas12_IS6110_cr5 & |
| | DNA; IDT | TCCACGCCGCCAA (SEQ ID NO: 35) | Cas13_IS6110_C |
| IS6110_cr6_F | custom | AGCTCGGCCGCGAAGAAAGCCGACGC | Cas12_IS6110_cr6 |
| | DNA; IDT | GGTC (SEQ ID NO: 36) | |
| IS6110_cr6_R | custom | TGAAGCGCTTGCGGCGGGACAACGCCG | Cas12_IS6110_cr6 |
| | DNA; IDT | AAT (SEQ ID NO: 37) | |
| IS1081_cr1_F | custom | ACCTCTCGGTTGAGGCGTTCCTGGGGG | Cas12_IS1081_cr1 |
| | DNA; IDT | TTGTTGGACCAGA (SEQ ID NO: 38) | |
| IS1081_cr1_R | custom | ACCTCGACACCGCCCGCACCGACCTGC | Cas12_IS1081_cr1 |
| | DNA; IDT | TGGCGTTCACCGC (SEQ ID NO: 39) | |
| IS1081_cr3_F | custom | GGTCCGAAACGCCTCTACGGCTTCGTC | Cas12_IS1081_cr3 |
| | DNA; IDT | GAGCTCTT (SEQ ID NO: 40) | |
| IS1081_cr3_R | custom | CCTGGTCGAAACACTTGGTGTGACAAA | Cas12_IS1081_cr3 |
| | DNA; IDT | GCTTTCCA (SEQ ID NO: 41) | |
| IS1081_cr4_F | custom | TTTGGCCATGATCGACACTTGCGACTT | Cas12_IS1081_cr4 |
| | DNA; IDT | GGAAAGC (SEQ ID NO: 42) | |
| IS1081_cr4_R | custom | ACCTGCTGGGAGTATCCACTCGCCGGA | Cas12_IS1081_cr4 |
| | DNA; IDT | TGGAGCGC (SEQ ID NO: 43) | |
| C13_FwT7_IS6_ | custom | gaaatTAATACGACTCACTATAgggGTCCC | Cas13_IS6110_ |
| A | DNA; IDT | GGACAGGCCGAGTTTGGTCATCAGCCG | A |
| | | TT (SEQ ID NO: 44) | |
| C13_FwT7_IS6_ | custom | gaaatTAATACGACTCACTATAgggTCGAT | Cas13_IS6110_ |
| B | DNA; IDT | GGACCGCCAGGGCTTGCCGGGTTTGAT | B |
| | | CA (SEQ ID NO: 45) | |
| C13_FwT7_IS6_ | custom | gaaatTAATACGACTCACTATAgggCCACC | Cas13_IS6110_ |
| sg5 | DNA; IDT | TCGATGCCCTCACGGTTCAGGGTTAGC | C |
| | | CA (SEQ ID NO: 46) | |
| C13_FwT7_IS1_ | custom | gaaatTAATACGACTCACTATAgggCGCCT | Cas13_IS1081_ |
| A | DNA; IDT | CTACGGCTTCGTCGAGCTCTTTGGCCAT | A |
| | | G (SEQ ID NO: 47) | |
| C13_FwT7_IS1_ | custom | gaaatTAATACGACTCACTATAgggCCAGC | Cas13_IS1081_ |
| B | DNA; IDT | AGGTAGCAGGTCGCCACCACGCTGGTC | B |
| | | AG (SEQ ID NO: 48) | |
| C13_FwT7_IS1_ | custom | gaaatTAATACGACTCACTATAgggTCACG | Cas13_IS1081_ |
| C | DNA; IDT | GTGGCGGTAGCCGTTGCGCTGATTGGA | C |
| | | CC (SEQ ID NO: 49) | |
| C13_Rv_IS6_A | custom | TTACGGTGCCCGCAAAGTGTGGCTAAC | Cas13_IS6110_ |
| | DNA; IDT | CCTGAACCGTGAG (SEQ ID NO: 50) | A |
| C13_Rv_IS6_B | custom | TCGGAGCGGTCGGAAGCTCCTATGACA | Cas13_IS6110_ |
| | DNA; IDT | ATGCACTAGCCGA (SEQ ID NO: 51) | B |
| C13_Rv_IS6_B | Custom | GCGAACTCAAGGAGCACATCAGCCGCG | Cas13_IS6110_ |
| | DNA; IDT | TCCACGCCGCCAA (SEQ ID NO: 52) | C |
| C13_Rv_IS1_A | custom | ACTCGCCGGATGGAGCGCCTGGTCGAA | Cas13_IS1081_ |
| | DNA; IDT | ACACTTGGTGTGA (SEQ ID NO: 53) | A |
| C13_Rv_IS1_B | custom | CCAAGCTGCGCCAGGGCAGCTATTTCC | Cas13_IS1081_ |
| | DNA; IDT | CGGACTGGCTGCT (SEQ ID NO: 54) | B |
| C13_Rv_IS1_C | custom | TTCATCGCCGCCTTGATGGGGGCTGAA | Cas13_IS1081_ |
| | DNA; IDT | GCCGACGCCCTGT (SEQ ID NO: 55) | C |
| LR5_1_FW | custom | gaaacatgtgctgtgtcaactcagggttaaatggattaag | Cas12_ |
| | DNA; IDT | (SEQ ID NO: 56) | internalControl_1 |
| LR5_1_RV | custom | tggtgatgactcttaaggagcatgctgccttcaagcatct | Cas12_ |
| | DNA; IDT | (SEQ ID NO: 57) | internalControl_1 |
| LR5_2_FW | custom | tggattaagggcggtgcaagatgtgctttgttaaacagat | Cas12_ |
| | DNA; IDT | (SEQ ID NO: 58) | internalControl_2 |
| LR5_2_RV | custom | tttgtgtccctgggtacttgagattagggagtggtgatga | Cas12_ |
| | DNA; IDT | (SEQ ID NO: 59) | internalControl_2 |
| LR5_3_FW | custom | gggcagcaatactgctttgtaaagcattgagatgtttatg | Cas12_ |
| | DNA; IDT | (SEQ ID NO: 60) | internalControl_3 |
| LR5_3_RV | custom | caaacacgtgaacaaaggtctttgcatcatagacaaggta | Cas12_ |
| | DNA; IDT | (SEQ ID NO: 61) | internalControl_3 |
| FAM-5C-Q | custom | /56-FAM/CCCCC/3IABkFQ/ (SEQ ID NO: 62) | Cas12 |
| | DNA; IDT | | fluorescent |
| | | | assays |
| HEX-5C-Q | custom | /5HEX/CCCCC/3IABkFQ/ (SEQ ID NO: 62) | Cas12-13 dual |
| | DNA; IDT | | fluorescence |
| | | | assays |
| FAM-6U-Q | custom | /56-FAM/rUrUrUrUrUrU/3IABkFQ/ (SEQ ID | Cas13 and |
| | RNA; IDT | NO: 63) | Cas12-13 |
| | | | fluorescence |
| | | | assays |
| FAM-14U-Bio | custom | /56-FAM/rUrUrUrUrUrUrUrUrUrUrUrUrUrU/ | Cas13 LFAs |
| | RNA; IDT | 3Bio/ (SEQ ID NO: 64) | |
| FAM-5C-Bio | custom | /56-FAM/CCCCC/3Bio/ (SEQ ID NO: 62) | Cas12 LFAs |
| | DNA; IDT | | |
| FAM-5C-Dig | custom | /56-FAM/CCCCC/3Dig_N/ (SEQ ID NO: 62) | Cas12 LFAs |
| | DNA; IDT | | |
| Bio-14C-FAM | custom | /5Biosg/CCCCCCCCCCCCCC/36-FAM/ | Cas12 LFAs |
| | DNA; IDT | (SEQ ID NO: 65) | |
| FAM-14C-Dig | custom | /56-FAM/CCCCCCCCCCCCCC/3Dig_N/ | Cas12 LFAs |
| | DNA; IDT | (SEQ ID NO: 65) | |
| | | | Relevant |
| | | | Figures |
| IS6110_A_target | gBlocks | GAACCGGATCGATGTGTACTGAGATCC | 7A |
| | gene | CCTATCCGTATGGTGGATAACGTCTTTC | |
| | fragments; | AGGTCGAGTACGCCTTCTTGTTGGCGGG | |
| | IDT | TCCAGATGGCTTGCTCGATCGCGTCGAG | |
| | | GACCATGGAGGTGGCCATCGTGGAAGC | |
| | | GACCCGCCAGCCCAGGATCCTGCGAGC | |
| | | GTAGGCGTCGGTGACAAAGGCCACGTA | |
| | | GGCGAACCCTGCCCAGGTCGACACATA | |
| | | GGTGAGGTCTGCTACCCACAGCCGGTT | |
| | | AGGTGCTGGTGGTCCGAAGCGGCGCTG | |
| | | GACGAGATCGGCGGGACGGGCTGTGGC | |
| | | CGGATCAGCGATCGTGGTCCTGCGGGC | |
| | | TTTGCCGCGGGTGGTCCCGGACAGGCC | |
| | | GAGTTTGGTCATCAGCCGTTCGACGGTG | |
| | | CATCTGGCCACCTCGATGCCCTCACGGT | |
| | | TCAGGGTTAGCCACACTTTGCGGGCACC | |
| | | GTAAACACCGTAGTTGGCGGCGTGGAC | |
| | | GCGGCTGATGTGCTCCTTGAGTTCGCCA | |
| | | TCGCGCAGCTCGCGGCGGCTGGGCTCC | |
| | | CGGTTGATGTGGTCGTAGTAGGTCGATG | |
| | | GGGCGATCGGCACACCCAGCTCGGTCA | |
| | | GCTGTGTGCAGATCGACTCGACACCCC | |
| | | ACCGCAAACCATCGGGGCCCTCGCGGT | |
| | | GGCCCTGATGATCGGCGATGAACCGGG | |
| | | TAATTAGCGTGCTGGCCGGTCGAGCTCG | |
| | | GCCGCGAAGAAAGCCGACGCGGTCTTT | |
| | | AAAATCGCGTTCGCCCTTCGCAATTCGG | |
| | | CGTTGTCCCGCCGCAAGCGCTTCAGCTC | |
| | | AGCGGATTCTTCGGTCGTGGTC (SEQ ID | |
| | | NO: 66) | |
| IS6110_B_target | gBlocks | AACCAGTCGACCCAGCGCGCGGTGGCC | 7A |
| | gene | AACTCGACATCCTCGATGGACCGCCAG | |
| | fragments; | GGCTTGCCGGGTTTGATCAGCTCGGTCT | |
| | IDT | TGTATAGGCCGTTGATCGTCTCGGCTAG | |
| | | TGCATTGTCATAGGAGCTTCCGACCGCT | |
| | | CCGACCGACGGTT (SEQ ID NO: 67) | |
| IS1081_C,B_ | gBlocks | TCCGGCGAGTGGATACTCCCAGCAGGT | 6B, 6C and 6D; |
| target | gene | AGCAGGTCGCCACCACGCTGGTCAGTG | 7A and 7B |
| | fragments; | CGCGTTCAGCTCGCTTGCGGCGCTGCAG | |
| | IDT | CAGCCAGTCCGGGAAATAGCTGCCCTG | |
| | | GCGCAGCTTGGGGATCGCGACGTCGAT | |
| | | GGTTGCGGCACGGGTGTCGAAATCACG | |
| | | GTGGCGGTAGCCGTTGCGCTGATTGGA | |
| | | CCGCTCATCGCTGCGTTCGCGGTAGCCC | |
| | | GCCCCGCACAGGGCGTCGGCTTCAGCC | |
| | | CCCATCAAGGCGGCGATGAACGTCGAG | |
| | | AGCAGCCCGC (SEQ ID NO: 68) | |
| LR5_target_3 | gBlocks | ggtgggacatgcgggcagcaatactgctttgtaaagcattgagat | 8B |
| | gene | gtttatgtgtatgcatatctaaaagcacagcacttaatcctttacctt | |
| | fragments; | gtctatgatgcaaagacctttgttcacgtgtttgtctgctga (SEQ | |
| | IDT | ID NO: 69) | |
| LR5_target_1-2 | gBlocks | cccgtgctctctgaaacatgtgctgtgtcaactcagggttaaatgg | |
| | gene | attaagggcggtgcaagatgtgctttgttaaacagatgcttgaag | |
| | fragments; | gcagcatgctccttaagagtcatcaccactccctaatctcaagtac | |
| | IDT | ccagggacacaaaaactgcggaagg (SEQ ID NO: 70) | |

Lower case “r” indicates RNA nucleotides.

TABLE 9
Optimized sequences used in Examples 5-10.

| | Oligo | | Gene | |
|---|---|---|---|---|
| | type; | | assay | |
| Name | company | Sequence | name | Figure |
| C13_IS6110_ | custom | /AltR1/rGrArUrUrUrArGrArCrUr | Cas13_ | FIGS. |
| sg5 | RNA; IDT | ArCrCrCrCrArArArArArCrGrArA | IS6110_C | 6A-6F |
| | | rGrGrGrGrArCrUrArArArArCrCr | | |
| | | UrArCrGrGrUrGrUrUrUrArCrGrG | | |
| | | rUrGrCrCrCrGrCrArArArGrUrG/ | | |
| | | AltR2/ (SEQ ID NO: 71) | | |
| C13_IS1081_ | custom | /AltR1/rGrArUrUrUrArGrArCrUr | Cas13_ | FIGS. |
| A | RNA; IDT | ArCrCrCrCrArArArArArCrGrArA | IS1081_A | 6A-6F |
| | | rGrGrGrGrArCrUrArArArArCrCr | | |
| | | ArArArGrCrUrUrUrCrCrArArGrU | | |
| | | rCrGrCrArArGrUrGrUrCrGrArU/ | | |
| | | AltR2/ (SEQ ID NO: 72) | | |
| IS6110_cr5 | custom | /AltR1/rUrArArUrUrUrCrUrArCr | Cas12_ | FIGS. |
| | RNA; IDT | UrArArGrUrGrUrArGrArUrCrGrG | IS6110_cr5 | 7A-7D |
| | | rGrCrArCrCrGrUrArArArCrArCrC | | |
| | | rGrUrA/AltR2/ (SEQ ID NO: 73) | | |
| LR5_crRNA_ | custom | /AltR1/rUrArArUrUrUrCrUrArCr | Cas12_ | FIGS. |
| 3 | RNA; IDT | UrArArGrUrGrUrArGrArUrArArA | internalControl_ | 8A-8G |
| | | rGrCrArCrArGrCrArCrUrUrArAr | 3 | |
| | | UrCrCrU/AltR2/ (SEQ ID NO: 74) | | |
| C13_FwT7_ | custom | gaaatTAATACGACTCACTATAgg | Cas13_ | FIGS. |
| IS6_sg5 | DNA; | gCCACCTCGATGCCCTCACGG | IS6110_C | 6A-6F |
| | IDT | TTCAGGGTTAGCCA (SEQ ID | | |
| | | NO: 75) | | |
| IS6110_ | custom | GCGAACTCAAGGAGCACATC | Cas12_ | FIGS. |
| cr5_R | DNA; | AGCCGCGTCCACGCCGCCAA | IS6110_cr5 & | 6A-6F |
| | IDT | (SEQ ID NO: 76) | Cas13_ | and |
| | | | IS6110_C | FIGS. |
| | | | | 7A-7D |
| C13_FwT7_ | custom | gaaatTAATACGACTCACTATAgg | Cas13_ | FIGS. |
| IS1_A | DNA; | gCGCCTCTACGGCTTCGTCGA | IS1081_A | 6A-6F |
| | IDT | GCTCTTTGGCCATG (SEQ ID | | |
| | | NO: 77) | | |
| C13_Rv_ | custom | ACTCGCCGGATGGAGCGCCTG | Cas13_ | FIGS. |
| IS1_A | DNA; | GTCGAAACACTTGGTGTGA | IS1081_A | 6A-6F |
| | IDT | (SEQ ID NO: 78) | | |
| IS6110_ | custom | TGGCCACCTCGATGCCCTCAC | Cas12_ | FIGS. |
| cr5_F | DNA; | GGTTCAGGGTTAGCCACAC | IS6110_cr5 | 7A-7D |
| | IDT | (SEQ ID NO: 79) | | |
| LR5_3_FW | custom | gggcagcaatactgctttgtaaagca | Cas12_ | FIGS. |
| | DNA; | ttgagatgtttatg (SEQ ID NO: 80) | internalControl_ | 8A-8G |
| | IDT | | 3 | |
| LR5_3_RV | custom | caaacacgtgaacaaaggtctttgca | Cas12_ | FIGS. |
| | DNA; | tcatagacaaggta (SEQ ID NO: 81) | internalControl_ | 8A-8G |
| | IDT | | 3 | |

Lower case “r” indicates RNA nucleotides.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated by reference in their entireties.

What is claimed is:

1. A dual assay for detecting a pathogen in a sample, comprising a first Cas-based assay and a second Cas-based assay comprising:
amplifying a target nucleic acid in the sample using recombinase polymerase amplification (RPA) to obtain an RPA product;
performing the first Cas-based assay by mixing a first Cas protein and a first CRISPR RNA (crRNA) with the RPA product in a first container; and
performing the second Cas-based assay by mixing a second Cas protein and a second crRNA with the RPA product in a second container.

2. The dual assay of claim 1, wherein the target nucleic acid comprises at least two different nucleic acid sequences.

3. The dual assay of claim 1, wherein the target nucleic acid comprises DNA or RNA.

4. The dual assay of claim 1, wherein the target nucleic acid comprises Mycobacterium tuberculosis (Mtb) insertion element IS6110 or IS1081.

5. The dual assay of claim 1, wherein the first Cas-based assay and the second Cas-based assay are performed in parallel.

6. The dual assay of claim 1, wherein the first Cas protein and the second Cas protein is Cas12a or Cas13a.

7. The dual assay of claim 1, wherein the pathogen comprises Mtb.

8. The dual assay of claim 1, comprising analyzing products of the first Cas-based assay and the second Cas-based assay using a lateral flow assay or a fluorescence-based assay.

9. The dual assay of claim 1, wherein the first Cas protein, the second Cas protein, the first crRNA, or the second crRNA is lyophilized.

10. The dual assay of claim 1, wherein the sample comprises sputum or saliva.

11. The dual assay of claim 1, wherein detection of the pathogen is completed within 90 minutes or less.

12. The dual assay of claim 1, wherein the target nucleic acid is obtained by:
a) adding a chelating agent to chelate polyvalent metal ions in the sample and a lysis agent to lyse two or more pathogens in the sample;
b) vortexing the sample;
c) heating the sample at about 95° C. for at least 30 minutes; and
d) spinning the sample to collect supernatant containing the nucleic acid, wherein the target nucleic acid is obtained in 15 minutes or less.

13. A kit comprising:
a container comprising a set of recombinase polymerase amplification (RPA) primers, a recombinase, and a crowding agent, wherein the container is adapted to receive a target nucleic acid;
two different containers, each of which comprises a Cas protein and a CRISPR RNA (crRNA), wherein the two containers are adapted to perform a Cas-based assay; and
optionally, instructions for use.

14. A method of co-extracting nucleic acid from two or more pathogens in a sample from a subject infected or suspected of being infected by the two or more pathogens, comprising:
a) adding a chelating agent to chelate polyvalent metal ions in the sample and a lysis agent to lyse the two or more pathogens in the sample;
b) vortexing the sample;
c) heating the sample at about 95° C. for at least 30 minutes; and
d) spinning the sample to collect supernatant containing the nucleic acid, wherein the method is completed in 15 minutes or less.

15. The method of claim 14, wherein the two or more pathogens comprise Mycobacterium tuberculosis (Mtb) and SARS-CoV-2 (SC2).

16. The method of claim 14, wherein the lysis agent comprises lysing matrix B beads.

17. The method of claim 14, wherein the chelating agent comprises Chelex-100 resin.

18. The method of claim 14, wherein the nucleic acid comprises RNA and DNA.

19. The method of claim 14, wherein the supernatant is analyzed with a diagnostic test.

20. The method of claim 14, wherein the sample comprises saliva or sputum.


SEQUENCE LISTING (81 sequences)

SEQ ID NO: 1 - DNA, 22 bp, synthetic construct
  source 1..22: mol_type = other DNA
cgccgcttcggaccaccagcac

SEQ ID NO: 2 - DNA, 25 bp, synthetic construct
  source 1..25: mol_type = other DNA
gtgacaaaggccacgtaggcgaacc

SEQ ID NO: 3 - DNA, 24 bp, synthetic construct
  source 1..24: mol_type = other DNA
cggctgtgggtagcagacctcacc

SEQ ID NO: 4 - DNA, 22 bp, synthetic construct
  source 1..22: mol_type = other DNA
ctaataccggataggaccacgg

SEQ ID NO: 5 - DNA, 22 bp, synthetic construct
  source 1..22: mol_type = other DNA
ctcatcccacaccgctaaagcg

SEQ ID NO: 6 - DNA, 34 bp, synthetic construct
  source 1..34: mol_type = other DNA
cgctccccttcgttcgcacggtgtcgctggagcg

SEQ ID NO: 7 - DNA, 20 bp, synthetic construct
  source 1..20: mol_type = other DNA
gaccccaaaatcagcgaaat

SEQ ID NO: 8 - DNA, 24 bp, synthetic construct
  source 1..24: mol_type = other DNA
tctggttactgccagttgaatctg

SEQ ID NO: 9 - DNA, 24 bp, synthetic construct
  source 1..24: mol_type = other DNA
accccgcattacgtttggtggacc

SEQ ID NO: 10 - RNA, 41 nt, synthetic construct
  source 1..41: mol_type = other RNA
  modified_base 1: mod_base = OTHER; note = uracil
  modified_base 4..6: mod_base = OTHER; note = uracil
  modified_base 8: mod_base = OTHER; note = uracil
  modified_base 11: mod_base = OTHER; note = uracil
  modified_base 15: mod_base = OTHER; note = uracil
  modified_base 17: mod_base = OTHER; note = uracil
  modified_base 21: mod_base = OTHER; note = uracil
  modified_base 23: mod_base = OTHER; note = uracil
  modified_base 36..37: mod_base = OTHER; note = uracil
  modified_base 41: mod_base = OTHER; note = uracil
taatttctactaagtgtagatctacccacagccggttaggt

SEQ ID NO: 11 - RNA, 41 nt, synthetic construct
  source 1..41: mol_type = other RNA
  modified_base 1: mod_base = OTHER; note = uracil
  modified_base 4..6: mod_base = OTHER; note = uracil
  modified_base 8: mod_base = OTHER; note = uracil
  modified_base 11: mod_base = OTHER; note = uracil
  modified_base 15: mod_base = OTHER; note = uracil
  modified_base 17: mod_base = OTHER; note = uracil
  modified_base 21: mod_base = OTHER; note = uracil
  modified_base 28: mod_base = OTHER; note = uracil
  modified_base 30: mod_base = OTHER; note = uracil
  modified_base 34: mod_base = OTHER; note = uracil
taatttctactaagtgtagataccggctgtgggtagcagac

SEQ ID NO: 12 - RNA, 41 nt, synthetic construct
  source 1..41: mol_type = other RNA
  modified_base 1: mod_base = OTHER; note = uracil
  modified_base 4..6: mod_base = OTHER; note = uracil
  modified_base 8: mod_base = OTHER; note = uracil
  modified_base 11: mod_base = OTHER; note = uracil
  modified_base 15: mod_base = OTHER; note = uracil
  modified_base 17: mod_base = OTHER; note = uracil
  modified_base 21: mod_base = OTHER; note = uracil
  modified_base 32: mod_base = OTHER; note = uracil
  modified_base 37..39: mod_base = OTHER; note = uracil
  modified_base 41: mod_base = OTHER; note = uracil
taatttctactaagtgtagataaagaccgcgtcggctttct

SEQ ID NO: 13 - RNA, 41 nt, synthetic construct
  source 1..41: mol_type = other RNA
  modified_base 1: mod_base = OTHER; note = uracil
  modified_base 4..6: mod_base = OTHER; note = uracil
  modified_base 8: mod_base = OTHER; note = uracil
  modified_base 11: mod_base = OTHER; note = uracil
  modified_base 15: mod_base = OTHER; note = uracil
  modified_base 17: mod_base = OTHER; note = uracil
  modified_base 21: mod_base = OTHER; note = uracil
  modified_base 29: mod_base = OTHER; note = uracil
  modified_base 32: mod_base = OTHER; note = uracil
taatttctactaagtgtagatccgcgggtggtcccggacag

SEQ ID NO: 14 - RNA, 41 nt, synthetic construct
  source 1..41: mol_type = other RNA
  modified_base 1: mod_base = OTHER; note = uracil
  modified_base 4..6: mod_base = OTHER; note = uracil
  modified_base 8: mod_base = OTHER; note = uracil
  modified_base 11: mod_base = OTHER; note = uracil
  modified_base 15: mod_base = OTHER; note = uracil
  modified_base 17: mod_base = OTHER; note = uracil
  modified_base 21: mod_base = OTHER; note = uracil
  modified_base 31: mod_base = OTHER; note = uracil
  modified_base 40: mod_base = OTHER; note = uracil
taatttctactaagtgtagatcgggcaccgtaaacaccgta

SEQ ID NO: 15 - RNA, 41 nt, synthetic construct
  source 1..41: mol_type = other RNA
  modified_base 1: mod_base = OTHER; note = uracil
  modified_base 4..6: mod_base = OTHER; note = uracil
  modified_base 8: mod_base = OTHER; note = uracil
  modified_base 11: mod_base = OTHER; note = uracil
  modified_base 15: mod_base = OTHER; note = uracil
  modified_base 17: mod_base = OTHER; note = uracil
  modified_base 21: mod_base = OTHER; note = uracil
  modified_base 25: mod_base = OTHER; note = uracil
  modified_base 30..31: mod_base = OTHER; note = uracil
  modified_base 37..38: mod_base = OTHER; note = uracil
taatttctactaagtgtagataaatcgcgttcgcccttcgc

SEQ ID NO: 16 - RNA, 41 nt, synthetic construct
  source 1..41: mol_type = other RNA
  modified_base 1: mod_base = OTHER; note = uracil
  modified_base 4..6: mod_base = OTHER; note = uracil
  modified_base 8: mod_base = OTHER; note = uracil
  modified_base 11: mod_base = OTHER; note = uracil
  modified_base 15: mod_base = OTHER; note = uracil
  modified_base 17: mod_base = OTHER; note = uracil
  modified_base 21: mod_base = OTHER; note = uracil
  modified_base 30: mod_base = OTHER; note = uracil
  modified_base 32: mod_base = OTHER; note = uracil
  modified_base 35..36: mod_base = OTHER; note = uracil
taatttctactaagtgtagatgcgccagatctgcttgggga

SEQ ID NO: 17 - RNA, 41 nt, synthetic construct
  source 1..41: mol_type = other RNA
  modified_base 1: mod_base = OTHER; note = uracil
  modified_base 4..6: mod_base = OTHER; note = uracil
  modified_base 8: mod_base = OTHER; note = uracil
  modified_base 11: mod_base = OTHER; note = uracil
  modified_base 15: mod_base = OTHER; note = uracil
  modified_base 17: mod_base = OTHER; note = uracil
  modified_base 21: mod_base = OTHER; note = uracil
  modified_base 26: mod_base = OTHER; note = uracil
  modified_base 29: mod_base = OTHER; note = uracil
  modified_base 36..37: mod_base = OTHER; note = uracil
taatttctactaagtgtagatgccatgatcgacacttgcga

SEQ ID NO: 18 - RNA, 41 nt, synthetic construct
  source 1..41: mol_type = other RNA
  modified_base 1: mod_base = OTHER; note = uracil
  modified_base 4..6: mod_base = OTHER; note = uracil
  modified_base 8: mod_base = OTHER; note = uracil
  modified_base 11: mod_base = OTHER; note = uracil
  modified_base 15: mod_base = OTHER; note = uracil
  modified_base 17: mod_base = OTHER; note = uracil
  modified_base 21..22: mod_base = OTHER; note = uracil
  modified_base 32: mod_base = OTHER; note = uracil
  modified_base 34..36: mod_base = OTHER; note = uracil
taatttctactaagtgtagattcacaccaagtgtttcgacc

SEQ ID NO: 19 - RNA, 64 nt, synthetic construct
  source 1..64: mol_type = other RNA
  modified_base 3..5: mod_base = OTHER; note = uracil
  modified_base 10: mod_base = OTHER; note = uracil
  modified_base 31: mod_base = OTHER; note = uracil
  modified_base 41: mod_base = OTHER; note = uracil
  modified_base 47: mod_base = OTHER; note = uracil
  modified_base 55: mod_base = OTHER; note = uracil
  modified_base 62: mod_base = OTHER; note = uracil
gatttagactaccccaaaaacgaaggggactaaaacggcatcgaggtggccagatgcaccgtcg

SEQ ID NO: 20 - RNA, 64 nt, synthetic construct
  source 1..64: mol_type = other RNA
  modified_base 3..5: mod_base = OTHER; note = uracil
  modified_base 10: mod_base = OTHER; note = uracil
  modified_base 31: mod_base = OTHER; note = uracil
  modified_base 42: mod_base = OTHER; note = uracil
  modified_base 51: mod_base = OTHER; note = uracil
  modified_base 53: mod_base = OTHER; note = uracil
gatttagactaccccaaaaacgaaggggactaaaacgacgatcaacggcctatacaagaccgag

SEQ ID NO: 21 - RNA, 64 nt, synthetic construct
  source 1..64: mol_type = other RNA
  modified_base 3..5: mod_base = OTHER; note = uracil
  modified_base 10: mod_base = OTHER; note = uracil
  modified_base 31: mod_base = OTHER; note = uracil
  modified_base 38: mod_base = OTHER; note = uracil
  modified_base 43: mod_base = OTHER; note = uracil
  modified_base 45..47: mod_base = OTHER; note = uracil
  modified_base 52: mod_base = OTHER; note = uracil
  modified_base 63: mod_base = OTHER; note = uracil
gatttagactaccccaaaaacgaaggggactaaaacctacggtgtttacggtgcccgcaaagtg

SEQ ID NO: 22 - RNA, 64 nt, synthetic construct
  source 1..64: mol_type = other RNA
  modified_base 3..5: mod_base = OTHER; note = uracil
  modified_base 10: mod_base = OTHER; note = uracil
  modified_base 31: mod_base = OTHER; note = uracil
  modified_base 43..45: mod_base = OTHER; note = uracil
  modified_base 51: mod_base = OTHER; note = uracil
  modified_base 58: mod_base = OTHER; note = uracil
  modified_base 60: mod_base = OTHER; note = uracil
  modified_base 64: mod_base = OTHER; note = uracil
gatttagactaccccaaaaacgaaggggactaaaaccaaagctttccaagtcgcaagtgtcgat

SEQ ID NO: 23 - RNA, 64 nt, synthetic construct
  source 1..64: mol_type = other RNA
  modified_base 3..5: mod_base = OTHER; note = uracil
  modified_base 10: mod_base = OTHER; note = uracil
  modified_base 31: mod_base = OTHER; note = uracil
  modified_base 55: mod_base = OTHER; note = uracil
gatttagactaccccaaaaacgaaggggactaaaacgcagcgccgcaagcgagctgaacgcgca

SEQ ID NO: 24 - RNA, 64 nt, synthetic construct
  source 1..64: mol_type = other RNA
  modified_base 3..5: mod_base = OTHER; note = uracil
  modified_base 10: mod_base = OTHER; note = uracil
  modified_base 31: mod_base = OTHER; note = uracil
  modified_base 43: mod_base = OTHER; note = uracil
  modified_base 60: mod_base = OTHER; note = uracil
gatttagactaccccaaaaacgaaggggactaaaacgcgggctaccgcgaacgcagcgatgagc

SEQ ID NO: 25 - RNA, 42 nt, synthetic construct
  source 1..42: mol_type = other RNA
  modified_base 1: mod_base = OTHER; note = uracil
  modified_base 4..6: mod_base = OTHER; note = uracil
  modified_base 8: mod_base = OTHER; note = uracil
  modified_base 11: mod_base = OTHER; note = uracil
  modified_base 15: mod_base = OTHER; note = uracil
  modified_base 17: mod_base = OTHER; note = uracil
  modified_base 21: mod_base = OTHER; note = uracil
  modified_base 32: mod_base = OTHER; note = uracil
  modified_base 34..35: mod_base = OTHER; note = uracil
taatttctactaagtgtagatacaaagcacatcttgcaccgc

SEQ ID NO: 26 - RNA, 42 nt, synthetic construct
  source 1..42: mol_type = other RNA
  modified_base 1: mod_base = OTHER; note = uracil
  modified_base 4..6: mod_base = OTHER; note = uracil
  modified_base 8: mod_base = OTHER; note = uracil
  modified_base 11: mod_base = OTHER; note = uracil
  modified_base 15: mod_base = OTHER; note = uracil
  modified_base 17: mod_base = OTHER; note = uracil
  modified_base 21: mod_base = OTHER; note = uracil
  modified_base 30: mod_base = OTHER; note = uracil
  modified_base 33: mod_base = OTHER; note = uracil
  modified_base 37..38: mod_base = OTHER; note = uracil
taatttctactaagtgtagataaggagcatgctgccttcaag

SEQ ID NO: 27 - RNA, 41 nt, synthetic construct
  source 1..41: mol_type = other RNA
  modified_base 1: mod_base = OTHER; note = uracil
  modified_base 4..6: mod_base = OTHER; note = uracil
  modified_base 8: mod_base = OTHER; note = uracil
  modified_base 11: mod_base = OTHER; note = uracil
  modified_base 15: mod_base = OTHER; note = uracil
  modified_base 17: mod_base = OTHER; note = uracil
  modified_base 21: mod_base = OTHER; note = uracil
  modified_base 34..35: mod_base = OTHER; note = uracil
  modified_base 38: mod_base = OTHER; note = uracil
  modified_base 41: mod_base = OTHER; note = uracil
taatttctactaagtgtagataaagcacagcacttaatcct

SEQ ID NO: 28 - DNA, 33 bp, synthetic construct
  source 1..33: mol_type = other DNA
cgtaggcgaaccctgcccaggtcgacacatagg

SEQ ID NO: 29 - DNA, 31 bp, synthetic construct
  source 1..31: mol_type = other DNA
cgatctcgtccagcgccgcttcggaccacca

SEQ ID NO: 30 - DNA, 30 bp, synthetic construct
  source 1..30: mol_type = other DNA
aattagcgtgctggccggtcgagctcggcc

SEQ ID NO: 31 - DNA, 30 bp, synthetic construct
  source 1..30: mol_type = other DNA
gggacaacgccgaattgcgaagggcgaacg

SEQ ID NO: 32 - DNA, 35 bp, synthetic construct
  source 1..35: mol_type = other DNA
tgtggccggatcagcgatcgtggtcctgcgggctt

SEQ ID NO: 33 - DNA, 35 bp, synthetic construct
  source 1..35: mol_type = other DNA
agatgcaccgtcgaacggctgatgaccaaactcgg

SEQ ID NO: 34 - DNA, 40 bp, synthetic construct
  source 1..40: mol_type = other DNA
tggccacctcgatgccctcacggttcagggttagccacac

SEQ ID NO: 35 - DNA, 40 bp, synthetic construct
  source 1..40: mol_type = other DNA
gcgaactcaaggagcacatcagccgcgtccacgccgccaa

SEQ ID NO: 36 - DNA, 30 bp, synthetic construct
  source 1..30: mol_type = other DNA
agctcggccgcgaagaaagccgacgcggtc

SEQ ID NO: 37 - DNA, 30 bp, synthetic construct
  source 1..30: mol_type = other DNA
tgaagcgcttgcggcgggacaacgccgaat

SEQ ID NO: 38 - DNA, 40 bp, synthetic construct
  source 1..40: mol_type = other DNA
acctctcggttgaggcgttcctgggggttgttggaccaga

SEQ ID NO: 39 - DNA, 40 bp, synthetic construct
  source 1..40: mol_type = other DNA
acctcgacaccgcccgcaccgacctgctggcgttcaccgc

SEQ ID NO: 40 - DNA, 35 bp, synthetic construct
  source 1..35: mol_type = other DNA
ggtccgaaacgcctctacggcttcgtcgagctctt

SEQ ID NO: 41 - DNA, 35 bp, synthetic construct
  source 1..35: mol_type = other DNA
cctggtcgaaacacttggtgtgacaaagctttcca

SEQ ID NO: 42 - DNA, 34 bp, synthetic construct
  source 1..34: mol_type = other DNA
tttggccatgatcgacacttgcgacttggaaagc

SEQ ID NO: 43 - DNA, 35 bp, synthetic construct
  source 1..35: mol_type = other DNA
acctgctgggagtatccactcgccggatggagcgc

SEQ ID NO: 44 - DNA, 59 bp, synthetic construct
  source 1..59: mol_type = other DNA
gaaattaatacgactcactataggggtcccggacaggccgagtttggtcatcagccgtt

SEQ ID NO: 45 - DNA, 59 bp, synthetic construct
  source 1..59: mol_type = other DNA
gaaattaatacgactcactatagggtcgatggaccgccagggcttgccgggtttgatca

SEQ ID NO: 46 - DNA, 59 bp, synthetic construct
  source 1..59: mol_type = other DNA
gaaattaatacgactcactatagggccacctcgatgccctcacggttcagggttagcca

SEQ ID NO: 47 - DNA, 59 bp, synthetic construct
  source 1..59: mol_type = other DNA
gaaattaatacgactcactatagggcgcctctacggcttcgtcgagctctttggccatg

SEQ ID NO: 48 - DNA, 59 bp, synthetic construct
  source 1..59: mol_type = other DNA
gaaattaatacgactcactatagggccagcaggtagcaggtcgccaccacgctggtcag

SEQ ID NO: 49 - DNA, 59 bp, synthetic construct
  source 1..59: mol_type = other DNA
gaaattaatacgactcactatagggtcacggtggcggtagccgttgcgctgattggacc

SEQ ID NO: 50 - DNA, 40 bp, synthetic construct
  source 1..40: mol_type = other DNA
ttacggtgcccgcaaagtgtggctaaccctgaaccgtgag

SEQ ID NO: 51 - DNA, 40 bp, synthetic construct
  source 1..40: mol_type = other DNA
tcggagcggtcggaagctcctatgacaatgcactagccga

SEQ ID NO: 52 - DNA, 40 bp, synthetic construct; identical to SEQ ID NO: 35
  source 1..40: mol_type = other DNA

SEQ ID NO: 53 - DNA, 40 bp, synthetic construct
  source 1..40: mol_type = other DNA
actcgccggatggagcgcctggtcgaaacacttggtgtga

SEQ ID NO: 54 - DNA, 40 bp, synthetic construct
  source 1..40: mol_type = other DNA
ccaagctgcgccagggcagctatttcccggactggctgct

SEQ ID NO: 55 - DNA, 40 bp, synthetic construct
  source 1..40: mol_type = other DNA
ttcatcgccgccttgatgggggctgaagccgacgccctgt

SEQ ID NO: 56 - DNA, 40 bp, synthetic construct
  source 1..40: mol_type = other DNA
gaaacatgtgctgtgtcaactcagggttaaatggattaag

SEQ ID NO: 57 - DNA, 40 bp, synthetic construct
  source 1..40: mol_type = other DNA
tggtgatgactcttaaggagcatgctgccttcaagcatct

SEQ ID NO: 58 - DNA, 40 bp, synthetic construct
  source 1..40: mol_type = other DNA
tggattaagggcggtgcaagatgtgctttgttaaacagat

SEQ ID NO: 59 - DNA, 40 bp, synthetic construct
  source 1..40: mol_type = other DNA
tttgtgtccctgggtacttgagattagggagtggtgatga

SEQ ID NO: 60 - DNA, 40 bp, synthetic construct
  source 1..40: mol_type = other DNA
gggcagcaatactgctttgtaaagcattgagatgtttatg

SEQ ID NO: 61 - DNA, 40 bp, synthetic construct
  source 1..40: mol_type = other DNA
caaacacgtgaacaaaggtctttgcatcatagacaaggta

SEQ ID NO: 62-63 - skipped (2 entries)

SEQ ID NO: 64 - RNA, 14 nt, synthetic construct
  source 1..14: mol_type = other RNA
  modified_base 1..14: mod_base = OTHER; note = uracil
tttttttttttttt

SEQ ID NO: 65 - DNA, 14 bp, synthetic construct
  source 1..14: mol_type = other DNA
cccccccccccccc

SEQ ID NO: 66 - DNA, 789 bp, synthetic construct
  source 1..789: mol_type = other DNA
gaaccggatcgatgtgtactgagatcccctatccgtatggtggataacgtctttcaggtcgagtacgccttcttgttggcgggtccagatggcttgctcgatcgcgtcgaggaccatggaggtggccatcgtggaagcgacccgccagcccaggatcctgcgagcgtaggcgtcggtgacaaaggccacgtaggcgaaccctgcccaggtcgacacataggtgaggtctgctacccacagccggttaggtgctggtggtccgaagcggcgctggacgagatcggcgggacgggctgtggccggatcagcgatcgtggtcctgcgggctttgccgcgggtggtcccggacaggccgagtttggtcatcagccgttcgacggtgcatctggccacctcgatgccctcacggttcagggttagccacactttgcgggcaccgtaaacaccgtagttggcggcgtggacgcggctgatgtgctccttgagttcgccatcgcgcagctcgcggcggctgggctcccggttgatgtggtcgtagtaggtcgatggggcgatcggcacacccagctcggtcagctgtgtgcagatcgactcgacaccccaccgcaaaccatcggggccctcgcggtggccctgatgatcggcgatgaaccgggtaattagcgtgctggccggtcgagctcggccgcgaagaaagccgacgcggtctttaaaatcgcgttcgcccttcgcaattcggcgttgtcccgccgcaagcgcttcagctcagcggattcttcggtcgtggtc

SEQ ID NO: 67 - DNA, 151 bp, synthetic construct
  source 1..151: mol_type = other DNA
aaccagtcgacccagcgcgcggtggccaactcgacatcctcgatggaccgccagggcttgccgggtttgatcagctcggtcttgtataggccgttgatcgtctcggctagtgcattgtcataggagcttccgaccgctccgaccgacggtt

SEQ ID NO: 68 - DNA, 282 bp, synthetic construct
  source 1..282: mol_type = other DNA
tccggcgagtggatactcccagcaggtagcaggtcgccaccacgctggtcagtgcgcgttcagctcgcttgcggcgctgcagcagccagtccgggaaatagctgccctggcgcagcttggggatcgcgacgtcgatggttgcggcacgggtgtcgaaatcacggtggcggtagccgttgcgctgattggaccgctcatcgctgcgttcgcggtagcccgccccgcacagggcgtcggcttcagcccccatcaaggcggcgatgaacgtcgagagcagcccgc

SEQ ID NO: 69 - DNA, 135 bp, synthetic construct
  source 1..135: mol_type = other DNA
ggtgggacatgcgggcagcaatactgctttgtaaagcattgagatgtttatgtgtatgcatatctaaaagcacagcacttaatcctttaccttgtctatgatgcaaagacctttgttcacgtgtttgtctgctga

SEQ ID NO: 70 - DNA, 162 bp, synthetic construct
  source 1..162: mol_type = other DNA
cccgtgctctctgaaacatgtgctgtgtcaactcagggttaaatggattaagggcggtgcaagatgtgctttgttaaacagatgcttgaaggcagcatgctccttaagagtcatcaccactccctaatctcaagtacccagggacacaaaaactgcggaagg

SEQ ID NO: 71 - RNA, 64 nt, synthetic construct; identical to SEQ ID NO: 21
  source 1..64: mol_type = other RNA
  modified_base 3..5: mod_base = OTHER; note = uracil
  modified_base 10: mod_base = OTHER; note = uracil
  modified_base 31: mod_base = OTHER; note = uracil
  modified_base 38: mod_base = OTHER; note = uracil
  modified_base 43: mod_base = OTHER; note = uracil
  modified_base 45..47: mod_base = OTHER; note = uracil
  modified_base 52: mod_base = OTHER; note = uracil
  modified_base 63: mod_base = OTHER; note = uracil

SEQ ID NO: 72 - RNA, 64 nt, synthetic construct; identical to SEQ ID NO: 22
  source 1..64: mol_type = other RNA
  modified_base 3..5: mod_base = OTHER; note = uracil
  modified_base 10: mod_base = OTHER; note = uracil
  modified_base 31: mod_base = OTHER; note = uracil
  modified_base 43..45: mod_base = OTHER; note = uracil
  modified_base 51: mod_base = OTHER; note = uracil
  modified_base 58: mod_base = OTHER; note = uracil
  modified_base 60: mod_base = OTHER; note = uracil
  modified_base 64: mod_base = OTHER; note = uracil

SEQ ID NO: 73 - RNA, 41 nt, synthetic construct; identical to SEQ ID NO: 14
  source 1..41: mol_type = other RNA
  modified_base 1: mod_base = OTHER; note = uracil
  modified_base 4..6: mod_base = OTHER; note = uracil
  modified_base 8: mod_base = OTHER; note = uracil
  modified_base 11: mod_base = OTHER; note = uracil
  modified_base 15: mod_base = OTHER; note = uracil
  modified_base 17: mod_base = OTHER; note = uracil
  modified_base 21: mod_base = OTHER; note = uracil
  modified_base 31: mod_base = OTHER; note = uracil
  modified_base 40: mod_base = OTHER; note = uracil

SEQ ID NO: 74 - RNA, 41 nt, synthetic construct; identical to SEQ ID NO: 27
  source 1..41: mol_type = other RNA
  modified_base 1: mod_base = OTHER; note = uracil
  modified_base 4..6: mod_base = OTHER; note = uracil
  modified_base 8: mod_base = OTHER; note = uracil
  modified_base 11: mod_base = OTHER; note = uracil
  modified_base 15: mod_base = OTHER; note = uracil
  modified_base 17: mod_base = OTHER; note = uracil
  modified_base 21: mod_base = OTHER; note = uracil
  modified_base 34..35: mod_base = OTHER; note = uracil
  modified_base 38: mod_base = OTHER; note = uracil
  modified_base 41: mod_base = OTHER; note = uracil

SEQ ID NO: 75 - DNA, 59 bp, synthetic construct; identical to SEQ ID NO: 46
  source 1..59: mol_type = other DNA

SEQ ID NO: 76 - DNA, 40 bp, synthetic construct; identical to SEQ ID NO: 35
  source 1..40: mol_type = other DNA

SEQ ID NO: 77 - DNA, 59 bp, synthetic construct; identical to SEQ ID NO: 47
  source 1..59: mol_type = other DNA

SEQ ID NO: 78 - DNA, 40 bp, synthetic construct; identical to SEQ ID NO: 53
  source 1..40: mol_type = other DNA

SEQ ID NO: 79 - DNA, 40 bp, synthetic construct; identical to SEQ ID NO: 34
  source 1..40: mol_type = other DNA

SEQ ID NO: 80 - DNA, 40 bp, synthetic construct; identical to SEQ ID NO: 60
  source 1..40: mol_type = other DNA

SEQ ID NO: 81 - DNA, 40 bp, synthetic construct; identical to SEQ ID NO: 61
  source 1..40: mol_type = other DNA